(19) United States
(12) Patent Application Publication
(10) Pub. No.: US 2011/0306986 A1
(43) Pub. Date: Dec. 15, 2011

(54) SURGICAL ROBOT SYSTEM USING AUGMENTED REALITY, AND METHOD FOR CONTROLLING SAME

(76) Inventors: Min Kyu Lee, Gyeonggi-do (KR); Seung Wook Choi, Gyeonggi-do (KR); Jong Seok Won, Gyeonggi-do (KR); Sung Kwan Hong, Seoul (KR)

(21) Appl. No.: 13/203,180

(22) PCT Filed: Mar. 22, 2010
(86) PCT No.: PCT/KR10/01740

§ 371 Date: Aug. 24, 2011

(30) Foreign Application Priority Data
Mar. 24, 2009 (KR) 10-2009-0025067
May 19, 2009 (KR) 10-2009-0043756

(51) Int. Cl. A61B 19/00

(52) U.S. Cl. 606/130

(57) ABSTRACT

Disclosed are a surgical robot system using augmented reality or history information and a control method thereof. A master interface for a surgical robot is provided, where the master interface is configured to be mounted on a master robot, which is configured to control a slave robot having a robot arm. The interface includes: a screen display unit configured to display an endoscope picture corresponding to a picture signal provided from a surgical endoscope; one or more arm manipulation unit for respectively controlling the robot arm; and an augmented reality implementer unit configured to generate virtual surgical tool information according to a user manipulation on the arm manipulation unit for displaying a virtual surgical tool through the screen display unit. This makes it possible to display an actual surgical tool and a virtual surgical tool together using augmented reality and thus enables surgery in a facilitated manner.

### CROSS-REFERENCE TO RELATED APPLICATIONS

This application is the National Phase of PCT/KR2010/001740 filed on Mar. 22, 2010, which claims priority under 35 U.S.C. 119(a) to Patent Application No. 10-2009-0025067 filed in the Republic of Korea on Mar. 24, 2009, and Patent Application No. 10-2009-0043756 filed in the Republic of Korea on May 19, 2009, all of which are hereby expressly incorporated by reference into the present application.

### BACKGROUND

The present invention relates to surgery, more particularly to a surgical robot system using augmented reality or history information and a control method thereof.

A surgical robot refers to a robot that has the capability to perform a surgical action in the stead of a surgeon. The surgical robot may provide the advantages of accurate and precise movements compared to a human and of enabling remote surgery.

Some of the surgical robots currently under development around the globe include bone surgery robots, laparoscopic surgery robots, stereotactic surgery robots, etc. Here, a laparoscopic surgical robot is a robot that performs minimally invasive surgery using a laparoscope and a miniature surgical tool.

Laparoscopic surgery is a cutting-edge technique that involves perforating a hole of about 1 cm in the navel area and inserting a laparoscope, which is an endoscope for looking inside the abdomen. Further advances in this technique are expected in the future.

Current laparoscopes are mounted with computer chips and have been developed to the extent that magnified visuals can be obtained that are clearer than images seen with the naked eye, and when used with specially-designed laparoscopic surgical tools while looking at a monitor screen, any type of surgery is possible.

Moreover, despite the fact that its surgical range is almost equal to that of laparotomy surgery, laparoscopic surgery produces fewer complications than does laparotomy, enables treatment within a much shorter time after the procedure, and helps the surgery patient maintain his/her stamina or immune functions. As such, laparoscopic surgery is being established as the standard surgery for treating colorectal cancer, etc., in places such as America and Europe.

A surgical robot system is generally composed of a master robot and a slave robot. When the operator manipulates a controller (e.g. handle) equipped on the master robot, a surgical tool coupled to or held by a robot arm on the slave robot may be manipulated to perform surgery.

The master robot and the slave robot may be coupled by a communication network for network communication. Here, if the network communication speed is not sufficiently fast, quite some time may pass before a manipulation signal transmitted from the master robot is received by the slave robot and/or a laparoscopic visual transmitted from a laparoscope camera mounted on the slave robot is received by the master robot.

It is generally known that, in order to perform surgery using a master robot and a slave robot, the network communication speed between the two has to be within 150 ms. If the communication speed is delayed any further, the movement of the operator's hand and the movement of the slave robot as seen through a screen may not agree with each other, making it very difficult for the operator.

Also, if the network communication speed between the master robot and the slave robot is slow, the operator may perform surgery while being wary of or having to predict the movement of the slave robot seen on the screen. This may cause unnatural movements, and in extreme cases, may prevent normal surgery.

Also, the conventional surgical robot system was limited in that the operator had to manipulate the controller equipped on the master robot with a high level of concentration throughout the entire period of operating on the surgery patient. This may cause severe fatigue to the operator, and an imperfect operation due to lowered concentration may cause severe aftereffects to the surgery patient.

### SUMMARY

An aspect of the invention is to provide a surgical robot system using augmented reality and its control method, in which an actual surgical tool and a virtual surgical tool are displayed together using augmented reality so as to enable surgery in a facilitated manner.

Another aspect of the invention is to provide a surgical robot system using augmented reality and its control method, with which various information regarding the patient can be outputted during surgery.

Another aspect of the invention is to provide a surgical robot system using augmented reality and its control method, in which the method of displaying the surgery screen can be varied according to the network communication speed between the master robot and the slave robot, so as to enable surgery in a facilitated manner.

Another aspect of the invention is to provide a surgical robot system using augmented reality and its control method, in which images inputted through an endoscope, etc., is processed automatically so as to be capable of immediately notifying the operator of emergency situations.

Another aspect of the invention is to provide a surgical robot system using augmented reality and its control method, with which occurrences of contacting an organ, etc., due to a movement of the virtual surgical tool, etc., caused by a manipulation on the master robot can be sensed in real time for informing the operator, and with which the positional relationship between the virtual surgical tool and the organ can be perceived intuitively.

Another aspect of the invention is to provide a surgical robot system using augmented reality and its control method, with which the patient's relevant image data (e.g. CT image, MRI image, etc.) with respect to the surgical site can be presented in real time so as to enable surgery that utilizes various types of information.

Another aspect of the invention is to provide a surgical robot system using augmented reality and its control method, which allow compatibility and enable sharing between a learner and a trainer so as to maximize the training effect.

Another aspect of the invention is to provide a surgical robot system using augmented reality and its control method, with which the progress and results of an actual surgical procedure can be predicted by utilizing a 3-dimensionally modeled virtual organ.

Another aspect of the invention is to provide a surgical robot system using history information and its control method, which enable complete or partial automatic surgery using history information of a virtual surgery performed using a virtual organ, etc., so as to reduce the operator's fatigue and allow the operator to maintain concentration during normal surgery.

Another aspect of the invention is to provide a surgical robot system using history information and its control method, which enable an operator to quickly respond with manual surgery in cases where the progress results of automatic surgery differ from the progress results of virtual surgery or where an emergency situation occurs.

One aspect of the present invention provides a surgical robot system, a slave robot, and a master robot that use augmented reality.

According to an embodiment of the invention, a master interface for a surgical robot is provided, where the master interface is configured to be mounted on a master robot, which is configured to control a slave robot having a robot arm. The interface includes: a screen display unit configured to display an endoscope picture corresponding to a picture signal provided from a surgical endoscope; one or more arm manipulation unit for respectively controlling the robot arm; and an augmented reality implementer unit configured to generate virtual surgical tool information according to a user manipulation on the arm manipulation unit for displaying a virtual surgical tool through the screen display unit.

The surgical endoscope can include one or more of a laparoscope, a thoracoscope, an arthroscope, a rhinoscope, a cystoscope, a rectoscope, a duodenoscope, a mediastinoscope, and a cardioscope.

The master interface for a surgical robot can further include a manipulation signal generator unit configured to generate a manipulation signal according to the user manipulation for controlling the robot arm and to transmit the manipulation signal to the slave robot.

The master interface for a surgical robot can further include: a drive mode selector unit for designating a drive mode of the master robot; and a control unit configured to provide control such that one or more of the endoscope picture and the virtual surgical tool is displayed through the screen display unit in correspondence with the drive mode selected by the drive mode selector unit.

The control unit can provide control such that a mode indicator corresponding to the selected drive mode is displayed through the screen display unit. The mode indicator can be pre-designated to be one or more of a text message, a boundary color, an icon, and a background color.

The slave robot can further include a vital information measurement unit. The vital information, measured by the vital information measurement unit, can be displayed through the screen display unit.

The augmented reality implementer unit can include: a characteristic value computation unit configured to compute a characteristic value using one or more of the endoscope picture and position coordinate information of an actual surgical tool coupled to one or more robot arm; and a virtual surgical tool generator unit configured to generate virtual surgical tool information according to a user manipulation using the arm manipulation unit.

The characteristic value computed by the characteristic value computation unit can include one or more of the surgical endoscope's field of view, magnifying ratio, viewpoint, and viewing depth, and the actual surgical tool's type, direction, depth, and bent angle.

The augmented reality implementer unit can further include: a test signal processing unit configured to transmit a test signal to the slave robot and to receive a response signal in response to the test signal from the slave robot; and a delay time calculating unit configured to calculate a delay value for one or more of a network communication speed and a network communication delay time between the master robot and the slave robot by using a transmission time of the test signal and a reception time of the response signal.

The master interface can further include: a control unit configured to provide control such that one or more of the endoscope picture and the virtual surgical tool is displayed through the screen display unit. Here, the control unit can provide control such that only the endoscope picture is displayed through the screen display unit if the delay value is equal to or lower than a preset delay threshold value.

The augmented reality implementer unit can further include a distance computation unit, which may compute a distance value between an actual surgical tool and a virtual surgical tool displayed through the screen display unit, by using position coordinates of each of the surgical tools.

The virtual surgical tool generator unit can provide processing such that the virtual surgical tool is not displayed through the screen display unit if the distance value computed by the distance computation unit is equal to or below a preset distance threshold value.

The virtual surgical tool generator unit can perform processing of one or more of adjusting translucency, changing color, and changing contour thickness for the virtual surgical tool in proportion to the distance value computed by the distance computation unit.

The augmented reality implementer unit can further include a picture analyzer unit configured to extract feature information by way of image processing the endoscope picture displayed through the screen display unit. Here, the feature information can include one or more of the endoscope picture's color value for each pixel, and the actual surgical tool's position coordinates and manipulation shape.

The picture analyzer unit can output a warning request if an area or a number of pixels in the endoscope picture having a color value included in a preset color value range exceeds a threshold value. One or more of displaying a warning message through the screen display unit, outputting a warning sound through a speaker unit, and stopping a display of the virtual surgical tool can be performed in response to the warning request.

The master interface can further include a network verifying unit configured to verify a network communication status between the master robot and the slave robot by using position coordinate information of the actual surgical tool included in the characteristic value computed by the characteristic value computation unit and position coordinate information of the virtual surgical tool included in the virtual surgical tool information generated by the virtual surgical tool generator unit.

The master interface can further include a network verifying unit configured to verify a network communication status between the master robot and the slave robot by using position coordinate information of each of the actual surgical tool and the virtual surgical tool included in the feature information extracted by the picture analyzer unit.

The network verifying unit can further use one or more of a trajectory and manipulation type of each of the surgical tools for verifying the network communication status.

The network verifying unit can verify the network communication status by determining whether or not the position coordinate information of the virtual surgical tool agrees with the position coordinate information of the actual surgical tool stored beforehand within a tolerance range.

The network verifying unit can output a warning request if the position coordinate information of the virtual surgical tool does not agree with the position coordinate information of the actual surgical tool within a tolerance range. One or more of displaying a warning message through the screen display unit, outputting a warning sound through a speaker unit, and stopping a display of the virtual surgical tool can be performed in response to the warning request.

The augmented reality implementer unit can further include: a picture analyzer unit configured to extract feature information, which may contain zone coordinate information of a surgical site or an organ displayed through the endoscope picture, by way of image processing the endoscope picture displayed through the screen display unit; and an overlap processing unit, configured to determine by using the virtual surgical tool information and the zone coordinate information whether or not there is overlapping such that the virtual surgical tool is positioned behind the zone coordinate information, and configured to provide processing such that a portion of a shape of the virtual surgical tool where overlapping occurs is concealed if there is overlapping.

The augmented reality implementer unit can further include: a picture analyzer unit configured to extract feature information, which may contain zone coordinate information of a surgical site or an organ displayed through the endoscope picture, by way of image processing the endoscope picture displayed through the screen display unit; and a contact recognition unit, configured to determine by using the virtual surgical tool information and the zone coordinate information whether or not there is contact between the virtual surgical tool and the zone coordinate information, and configured to perform processing such that a contact warning is provided if there is contact.

The contact warning can include one or more of processing a force feedback, limiting a manipulation of the arm manipulation unit, displaying a warning message through the screen display unit, and outputting a warning sound through a speaker unit.

The master interface can further include: a storage unit storing a reference picture, which may include one or more of an X-ray picture, a computed tomography (CT) picture, and a magnetic resonance imaging (MRI) picture; and a picture analyzer unit configured to recognize a surgical site or an organ displayed through the endoscope picture, by way of image processing the endoscope picture displayed through the screen display unit. The reference picture can be displayed, in correspondence with a name of an organ recognized by the picture analyzer, on a display screen independent of the display screen on which the endo scope picture is displayed.

The master interface can further include: a storage unit storing a reference picture, which may include one or more of an X-ray picture, a computed tomography (CT) picture, and a magnetic resonance imaging (MRI) picture. The reference picture can be displayed, in correspondence with position coordinate information of the actual surgical tool computed by the characteristic value computation unit, on a display screen together with the endo scope picture or on a display screen independent of the display screen on which the endo scope picture is displayed.

The reference picture can be displayed as a 3-dimensional picture using MPR (multi-planar reformatting).

According to another embodiment of the invention, a surgical robot system is provided that includes: two or more master robots coupled to each other via a communication network; and a slave robot having one or more robot arm, which may be controlled according to a manipulation signal received from any of the master robots.

Each of the master robots can include: a screen display unit configured to display an endoscope picture corresponding to a picture signal provided from a surgical endoscope; one or more arm manipulation unit for respectively controlling the robot arm; and an augmented reality implementer unit configured to generate virtual surgical tool information according to a user manipulation on the arm manipulation unit for displaying a virtual surgical tool through the screen display unit.

A manipulation on an arm manipulation unit of a first master robot of the two or more master robots can serve to generate the virtual surgical tool information, and a manipulation on an arm manipulation unit of a second master robot of the two or more master robots can serve to control the robot arm.

A virtual surgical tool corresponding to the virtual surgical tool information according to a manipulation on the arm manipulation unit of the first master robot can be displayed through the screen display unit of the second master robot.

Another aspect of the present invention provides a method of controlling a surgical robot system and a method of operating a surgical robot system, as well as recorded media on which programs for implementing these methods are recorded, respectively.

According to an embodiment of the invention, a method of controlling a surgical robot system is provided, which is performed in a master robot configured to control a slave robot having a robot arm. The method includes: displaying an endoscope picture corresponding to a picture signal inputted from a surgical endoscope; generating virtual surgical tool information according to a manipulation on an arm manipulation unit; and displaying a virtual surgical tool corresponding to the virtual surgical tool information together with the endoscope picture.

The surgical endoscope can include one or more of a laparoscope, a thoracoscope, an arthroscope, a rhinoscope, a cystoscope, a rectoscope, a duodenoscope, a mediastinoscope, and a cardioscope.

Generating the virtual surgical tool information can include: receiving as input manipulation information according to a manipulation on the arm manipulation unit; and generating the virtual surgical tool information and a manipulation signal for controlling the robot arm according to the manipulation information. The manipulation signal can be transmitted to the slave robot for controlling the robot arm.

The method of controlling a surgical robot system can further include: receiving as input a drive mode selection command for designating a drive mode of the master robot; and providing control such that one or more of the endoscope picture and the virtual surgical tool are displayed through the screen display unit according to the drive mode selection command. The method can also further include providing control such that a mode indicator corresponding to the drive mode designated by the drive mode selection command is displayed through the screen display unit.

The mode indicator can be pre-designated to be one or more of a text message, a boundary color, an icon, and a background color.

The method of controlling a surgical robot system can further include: receiving vital information measured from the slave robot; and displaying the vital information in a display area independent of a display area on which the endoscope picture is displayed.

The method of controlling a surgical robot system can further include computing a characteristic value using one or more of the endoscope picture and position coordinate information of an actual surgical tool coupled to the robot arm. The characteristic value can include one or more of the surgical endoscope's field of view, magnifying ratio, viewpoint, and viewing depth, and the actual surgical tool's type, direction, depth, and bent angle.

The method of controlling a surgical robot system can further include: transmitting a test signal to the slave robot; receiving a response signal in response to the test signal from the slave robot; and calculating a delay value for one or more of a network communication speed and a network communication delay time between the master robot and the slave robot by using a transmission time of the test signal and a reception time of the response signal.

Displaying the virtual surgical tool together with the endoscope picture can include: determining whether or not the delay value is equal to or lower than a preset delay threshold value; providing processing such that the virtual surgical tool is displayed together with the endoscope picture, if the delay threshold value is exceeded; and providing processing such that only the endoscope picture is displayed, if the delay threshold value is not exceeded.

The method of controlling a surgical robot system can further include: computing position coordinates of the endoscope picture displayed including an actual surgical tool and the displayed virtual surgical tool; and computing a distance value between the respective surgical tools by using the position coordinates of the respective surgical tools.

Displaying the virtual surgical tool together with the endoscope picture can include: determining whether or not the distance value is equal to or lower than a preset distance threshold value; and providing processing such that the virtual surgical tool is displayed together with the endoscope picture only if the distance value is equal to or lower than the distance threshold value.

Also, displaying of the virtual surgical tool together with the endoscope picture can include: determining whether or not the distance value is equal to or lower than a preset distance threshold value; and providing processing such that the virtual surgical tool is displayed together with the endoscope picture, with one or more processing for adjusting translucency, changing color, and changing contour thickness applied to the virtual surgical tool, if the distance threshold value is exceeded.

The method of controlling a surgical robot system can further include: determining whether or not the position coordinates of each of the surgical tools agree with each other within a tolerance range; and verifying a communication status between the master robot and the slave robot from a result of the determining.

During the determining, it can be determined whether or not current position coordinates of the virtual surgical tool agree with previous position coordinates of the actual surgical tool within a tolerance range.

Also, during the determining, it can further be determined whether or not one or more of a trajectory and manipulation type of each of the surgical tools agree with each other within a tolerance range.

The method of controlling a surgical robot system can further include: extracting feature information, which may contain a color value for each pixel in the endoscope picture being displayed; determining whether or not an area or a number of pixels in the endoscope picture having a color value included in a preset color value range exceeds a threshold value, and outputting warning information if the threshold value is exceeded.

One or more of displaying a warning message, outputting a warning sound, and stopping a display of the virtual surgical tool can be performed in response to the warning information.

Displaying the virtual surgical tool together with the endoscope picture can include: extracting zone coordinate information of a surgical site or an organ displayed through the endoscope picture by way of image processing the endoscope picture; determining by using the virtual surgical tool information and the zone coordinate information whether or not there is overlapping such that the virtual surgical tool is positioned behind the zone coordinate information; and providing processing such that a portion of a shape of the virtual surgical tool where overlapping occurs is concealed, if there is overlapping.

The method of controlling a surgical robot system can further include: extracting zone coordinate information of a surgical site or an organ displayed through the endoscope picture by way of image processing the endoscope picture; determining by using the virtual surgical tool information and the zone coordinate information whether or not there is contact between the virtual surgical tool and the zone coordinate information; and performing processing such that a contact warning is provided, if there is contact.

Processing the contact warning can include one or more of processing a force feedback, limiting a manipulation of the arm manipulation unit, displaying a warning message, and outputting a warning sound.

The method of controlling a surgical robot system can include: recognizing a surgical site or an organ displayed through the endoscope picture, by way of image processing the endoscope picture; and extracting and displaying a reference picture of a position corresponding to a name of the recognized organ from among pre-stored reference pictures. Here, the reference picture can include one or more of an X-ray picture, a computed tomography (CT) picture, and a magnetic resonance imaging (MRI) picture.

The method of controlling a surgical robot system can include extracting a reference image corresponding to the position coordinates of the actual surgical tool from among pre-stored reference pictures; and extracting and displaying the extracted reference picture. The reference picture can include one or more of an X-ray picture, a computed tomography (CT) picture, and a magnetic resonance imaging (MRI) picture.

The reference picture can be displayed together on a display screen on which the endoscope picture is displayed or can be displayed through a display screen independent of the display screen on which the endoscope picture is displayed.

The reference picture can be displayed as a 3-dimensional picture using MPR (multi-planar reformatting).

According to another embodiment of the invention, a method of operating a surgical robot system is provided, for a surgical robot system including a slave robot having a robot arm and a master robot controlling the slave robot. The method includes: generating, by a first master robot, of virtual surgical tool information for displaying a virtual surgical tool in correspondence with a manipulation on an arm manipulation unit and of a manipulation signal for controlling the robot arm; and transmitting, by the first master robot, of the manipulation signal to the slave robot and of one or more of the manipulation signal and the virtual surgical tool information to a second master robot, where the second master robot displays a virtual surgical tool corresponding to one or more of the manipulation signal and the virtual surgical tool information through a screen display unit.

Each of the first master robot and the second master robot can display an endo scope picture received from the slave robot through a screen display unit, and the virtual surgical tool can be displayed together with the endoscope picture.

The method of operating a surgical robot system can further include: determining, by the first master robot, of whether or not a surgery authority retrieve command is received from the second master robot; and providing control, by the first master robot, such that a manipulation on the arm manipulation unit functions only to generate the virtual surgical tool information, if the surgery authority retrieve command is received.

According to yet another embodiment of the invention, a method of simulating surgery is provided, which may be performed at a master robot that controls a slave robot having a robot arm. The method includes: recognizing organ selection information; and displaying a 3-dimensional organ image corresponding to the organ selection information by using pre-stored organ modeling information, where the organ modeling information includes characteristic information of each point of an interior and an exterior of a corresponding organ, the characteristic information including one or more of a shape, color, and tactile feel.

Recognizing the organ selection information can be accomplished by: analyzing information on one or more of a color and an appearance of an organ included in a surgical site by using a picture signal inputted from a surgical endo scope; and recognizing an organ matching the analyzed information from among pre-stored organ modeling information.

The organ selection information can include one or more organ selected and inputted by an operator.

The method can also further include: receiving as input a surgical manipulation command for the 3-dimensional organ image according to a manipulation on an arm manipulation unit; and outputting tactile information according to the surgical manipulation command by using the organ modeling information.

The tactile information can include control information for controlling one or more of manipulation sensitivity and manipulation resistance with respect to the manipulation on the arm manipulation unit or control information for processing a force feedback.

The method can further include: receiving as input a surgical manipulation command for the 3-dimensional organ image according to a manipulation on an arm manipulation unit; and displaying a manipulation result image according to the surgical manipulation command by using the organ modeling information.

The surgical manipulation command can include one or more of incision, suturing, pulling, pushing, organ deformation due to contact, organ damage due to electrosurgery, and bleeding from a blood vessel.

The method can also further include: recognizing an organ according to the organ selection information; and extracting and displaying a reference picture of a position corresponding to a name of the recognized organ from among pre-stored reference pictures. Here, the reference picture can include one or more of an X-ray picture, a computed tomography (CT) picture, and a magnetic resonance imaging (MRI) picture.

Yet another aspect of the invention provides a master robot, which is configured to control a slave robot having a robot arm by using a manipulation signal, and which includes: a storage element; an augmented reality implementer unit configured to store a sequential user manipulation history for virtual surgery using a 3-dimensional modeling image in the storage element as surgical action history information; and a manipulation signal generator unit configured to transmit to the slave robot a manipulation signal generated using the surgical action history information, if an apply command is inputted.

The storage element can further store characteristic information related to an organ corresponding to the 3-dimensional modeling image, where the characteristic information can include one or more of a 3-dimensional image, interior shape, exterior shape, size, texture, and tactile feel during incision related to the organ.

The master robot can further include a modeling application unit configured to correct the 3-dimensional modeling image to be aligned with feature information recognized using a reference picture.

The storage element can further store the reference picture, which may include one or more of an X-ray picture, a computed tomography (CT) picture, and a magnetic resonance imaging (MRI) picture, and the surgical action history information can be renewed using a correction result of the modeling application unit.

The reference picture can be processed as a 3-dimensional picture using MPR (multi-planar reformatting).

The augmented reality implementer unit can determine whether or not a pre-designated anomaly exists in the user manipulation history, and if so, can renew the surgical action history information such that the anomaly is processed according to a pre-designated rule.

If the surgical action history information is composed such that a user manipulation is required while proceeding with automatic surgery, the generating of the manipulation signal can be stopped until a required user manipulation is inputted.

The surgical action history information can include a user manipulation history for one or more of an entire surgical procedure, a partial surgical procedure, and a unit action.

The master robot can further include a screen display unit, where vital information measured and provided by a vital information measurement unit of the slave robot can be displayed through the screen display unit.

Still another aspect of the invention provides a master robot, in a surgical robot system which includes the master robot and a slave robot, where the master robot is configured to control and monitor an action of the slave robot. The master robot includes: an augmented reality implementer unit configured to store a sequential user manipulation history for virtual surgery using a 3-dimensional modeling image in a storage element as surgical action history information and configured to further store progress information of the virtual surgery; a manipulation signal generator unit configured to transmit to the slave robot a manipulation signal generated using the surgical action history information, if an apply command is inputted; and a picture analyzer unit configured to determine whether or not analysis information and the progress information agree with each other within a pre-designated tolerance range, the analysis information obtained by analyzing a picture signal provided from a surgical endoscope of the slave robot.

The progress information and the analysis information can include one or more of a length, area, shape, and bleeding amount of an incision surface.

If the analysis information and the progress information do not agree with each other within the pre-designated tolerance range, the transmission of the manipulation signal can be stopped.

If the analysis information and the progress information do not agree with each other within a pre-designated tolerance range, the picture analyzer unit can output a warning request, and one or more of displaying a warning message through a screen display unit and outputting a warning sound through a speaker unit can be performed in response to the warning request.

The surgical endoscope can include one or more of a laparoscope, a thoracoscope, an arthroscope, a rhinoscope, a cystoscope, a rectoscope, a duodenoscope, a mediastinoscope, and a cardioscope.

The master robot can further include a screen display unit, where vital information measured and provided by a vital information measurement unit of the slave robot can be displayed through the screen display unit.

The storage element can further store characteristic information related to an organ corresponding to the 3-dimensional modeling image. Here, the characteristic information can include one or more of a 3-dimensional image, interior shape, exterior shape, size, texture, and tactile feel during incision related to the organ.

A modeling application unit can further be included, which may be configured to correct the 3-dimensional modeling image to be aligned with feature information recognized using a reference picture.

The storage element can further store the reference picture, which may include one or more of an X-ray picture, a computed tomography (CT) picture, and a magnetic resonance imaging (MRI) picture, and the surgical action history information can be renewed using a correction result of the modeling application unit.

The reference picture can be processed as a 3-dimensional picture using MPR (multi-planar reformatting).

The picture analyzer unit can output a warning request, if an area or a number of pixels in the endoscope picture having a color value included in a preset color value range exceeds a threshold value. One or more of displaying a warning message through a screen display unit and outputting a warning sound through a speaker unit can be performed in response to the warning request.

The picture analyzer unit can extract zone coordinate information of a surgical site or an organ displayed through an endoscope picture, by way of image processing the endoscope picture displayed through a screen display unit, in order to generate the analysis information.

Another aspect of the invention provides a method by which a master robot controls a slave robot having a robot arm by using a manipulation signal. This method includes: generating surgical action history information for a sequential user manipulation for virtual surgery using a 3-dimensional modeling image; determining whether or not an apply command is inputted; and generating a manipulation signal using the surgical action history information and transmitting the manipulation signal to the slave robot, if the apply command is inputted.

The method can further include: renewing using a reference picture such that characteristic information related to a corresponding organ is aligned with a pre-stored 3-dimensional modeling image; and correcting the surgical action history information to conform with the renewing result.

The characteristic information can include one or more of a 3-dimensional image, interior shape, exterior shape, size, texture, and tactile feel during incision related to the organ.

The reference picture can include one or more of an X-ray picture, a computed tomography (CT) picture, and a magnetic resonance imaging (MRI) picture.

The reference picture can be processed as a 3-dimensional picture using MPR (multi-planar reformatting).

The method can further include: determining whether or not a pre-designated anomaly exists in the sequential user manipulation; and renewing the surgical action history information such that the anomaly is processed according to a pre-designated rule, if the pre-designated anomaly exists in the sequential user manipulation.

During the generating and transmitting of the manipulation signal to the slave robot, if the surgical action history information is composed such that a user manipulation is required while proceeding with automatic surgery, the generating of the manipulation signal can be stopped until a required user manipulation is inputted.

The surgical action history information can include a user manipulation history for one or more of an entire surgical procedure, a partial surgical procedure, and a unit action.

Before the determining operation, the method can include: performing a virtual simulation using the generated surgical action history information, if a virtual simulation command is inputted; determining whether or not modification information for the surgical action history information is inputted; and renewing the surgical action history information using the inputted modification information, if the modification information is inputted.

Yet another aspect of the invention provides a method by which a master robot monitors an action of a slave robot, in a surgical robot system comprising the master robot and the slave robot. This method includes: generating surgical action history information for a sequential user manipulation for virtual surgery using a 3-dimensional modeling image, and generating progress information of the virtual surgery; generating a manipulation signal using the surgical action history information and transmitting the manipulation signal to the slave robot, if an apply command is inputted; generating analysis information by analyzing a picture signal provided from a surgical endoscope of the slave robot; and determining whether or not the analysis information and the progress information agree with each other within a pre-designated tolerance range.

The progress information and the analysis information can include one or more of a length, area, shape, and bleeding amount of an incision surface.

If the analysis information and the progress information do not agree with each other within the pre-designated tolerance range, the transmission of the manipulation signal can be stopped.

The method can further include outputting a warning request, if the analysis information and the progress information do not agree with each other within the pre-designated tolerance range. Here, one or more of displaying a warning message through a screen display unit and outputting a warning sound through a speaker unit can be performed in response to the warning request.

The surgical endoscope can include one or more of a laparoscope, a thoracoscope, an arthroscope, a rhinoscope, a cystoscope, a rectoscope, a duodenoscope, a mediastinoscope, and a cardioscope.

Characteristic information related to an organ corresponding to the 3-dimensional modeling image can be stored beforehand, and the characteristic information can include one or more of a 3-dimensional image, interior shape, exterior shape, size, texture, and tactile feel during incision related to the organ.

The 3-dimensional modeling image can be corrected to be aligned with feature information recognized using a reference picture.

The reference picture can include one or more of an X-ray picture, a computed tomography (CT) picture, and a magnetic resonance imaging (MRI) picture, and the surgical action history information can be renewed using a correction result of the modeling application unit.

The reference picture can be processed as a 3-dimensional picture using MPR (multi-planar reformatting).

The method can further include: determining whether or not an area or a number of pixels in an endo scope picture having a color value included in a preset color value range exceeds a threshold value; and outputting a warning request, if the area or number exceeds the threshold value. Here, one or more of displaying a warning message through a screen display unit and outputting a warning sound through a speaker unit can be performed in response to the warning request.

In order to generate the analysis information, zone coordinate information of a surgical site or an organ displayed through an endoscope picture can be extracted, by way of image processing the endoscope picture displayed through a screen display unit.

Additional aspects, features, and advantages, other than those described above, will be apparent from the drawings, claims, and written description below.

### BRIEF DESCRIPTION OF THE DRAWINGS

FIG. 1 is a plan view illustrating the overall structure of a surgical robot according to an embodiment of the invention.

FIG. 2 is a conceptual drawing illustrating the master interface of a surgical robot according to an embodiment of the invention.

FIG. 3 is a block diagram schematically illustrating the composition of a master robot and a slave robot according to an embodiment of the invention.

FIG. 4 illustrates an example of drive modes for a surgical robot system according to an embodiment of the invention.

FIG. 5 illustrates an example of a mode indicator showing an active drive mode according to an embodiment of the invention.

FIG. 6 is a flowchart illustrating a procedure of selecting a drive mode between a first mode and a second mode.

FIG. 7 illustrates an example of a screen display outputted through a monitor unit in the second mode according to an embodiment of the invention.

FIG. 8 illustrates the detailed composition of an augmented reality implementer unit according to an embodiment of the invention.

FIG. 9 is a flowchart illustrating a method of driving a master robot in the second mode according to an embodiment of the invention.

FIG. 10 illustrates the detailed composition of an augmented reality implementer unit according to another embodiment of the invention.

FIG. 11 and FIG. 12 are flowcharts respectively illustrating methods of driving a master robot in the second mode according to different embodiments of the invention.

FIG. 13 is a block diagram schematically illustrating the composition of a master robot and a slave robot according to yet another embodiment of the invention.

FIG. 14 is a flowchart illustrating a method of verifying normal driving of a surgical robot system according to yet another embodiment of the invention.

FIG. 15 illustrates the detailed composition of an augmented reality implementer unit according to yet another embodiment of the invention.

FIG. 16 and FIG. 17 are flowcharts respectively illustrating methods of driving a master robot for outputting a virtual surgical tool according to different embodiments of the invention.

FIG. 18 is a flowchart illustrating a method of providing a reference image according to yet another embodiment of the invention.

FIG. 19 is a plan view illustrating the overall structure of a surgical robot according to yet another embodiment of the invention.

FIG. 20 illustrates a method of operating a surgical robot system in training mode according to yet another embodiment of the invention.

FIG. 21 illustrates a method of operating a surgical robot system in training mode according to yet another embodiment of the invention.

FIG. 22 illustrates the detailed composition of an augmented reality implementer unit according to another embodiment of the invention.

FIG. 23 is a block diagram schematically illustrating the composition of a master robot and a slave robot according to yet another embodiment of the invention.

FIG. 24 illustrates the detailed composition of an augmented reality implementer unit 350 according to yet another embodiment of the invention.

FIG. 25 is a flowchart illustrating a method of automatic surgery using history information according to an embodiment of the invention.

FIG. 26 is a flowchart illustrating a procedure of renewing surgical action history information according to another embodiment of the invention.

FIG. 27 is a flowchart illustrating a method of automatic surgery using history information according to yet another embodiment of the invention.

FIG. 28 is a flowchart illustrating a method of monitoring surgery progress according to yet another embodiment of the invention.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added. Certain embodiments of the present invention will be described below in detail with reference to the accompanying drawings.

Although the spirit of the invention can be generally applied to surgical operations in which a surgical endoscope (e.g. a laparoscope, thoracoscope, arthroscope, rhinoscope, etc.) is used, the embodiments of the invention will be described, for convenience, using examples in which a laparoscope is used.

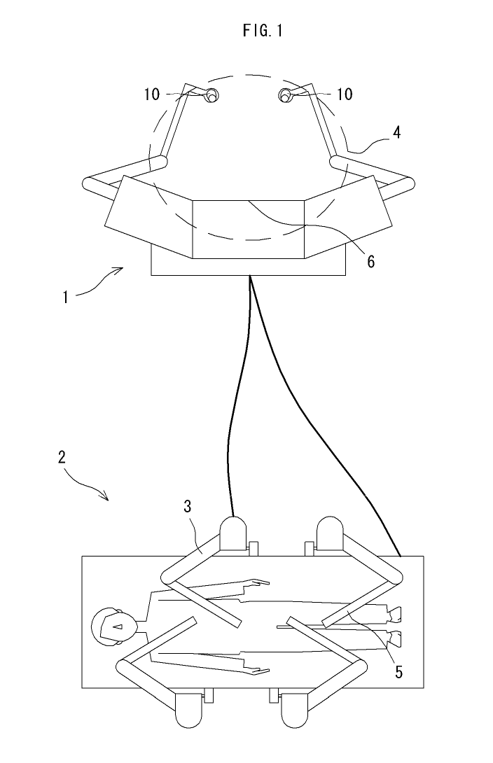

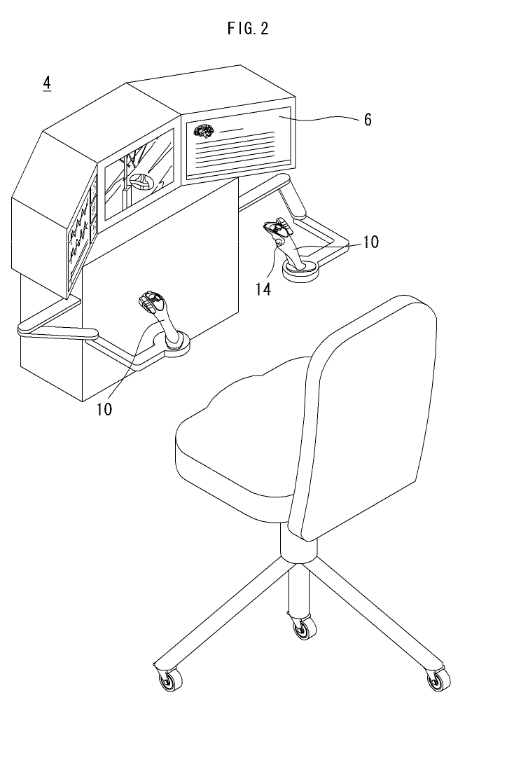

FIG. 1 is a plan view illustrating the overall structure of a surgical robot according to an embodiment of the invention, and FIG. 2 is a conceptual drawing illustrating the master interface of a surgical robot according to an embodiment of the invention.

Referring to FIG. 1 and FIG. 2, a robot system for laparoscopic surgery may include a slave robot 2, which performs surgery on a patient lying on the operating table, and a master robot 1, by which the operator remotely controls the slave robot 2. The master robot 1 and slave robot 2 do not necessarily have to be physically separated as independent individual devices, but can be integrated into a single body, in which case a master interface 4 can correspond, for instance, to the interface portion of the integrated robot.

The master interface 4 of the master robot 1 may include a monitor unit 6 and a master controller, while the slave robot 2 may include robot arms 3 and a laparoscope 5. The master interface 4 can further include a mode-changing control button. The mode-changing control button can be implemented in the form of a clutch button 14 or a pedal (not shown), etc., although the implementation of the mode-changing control button is not thus limited, and the mode-changing control button can also be implemented as mode a function menu or a selection menu displayed through the monitor unit 6. Also, the usage of the pedal, etc., can be set, for example, to perform any action required during a surgical procedure.

The master interface 4 may include master controllers, which may be held by both hands of the operator for manipulation. The master controller can be implemented as two handles 10 or more, as illustrated in FIG. 1 and FIG. 2, and a manipulation signal resulting from the operator's manipulation of the handles 10 may be transmitted to the slave robot 2 to control the robot arm 3. The operator's manipulation of the handles 10 can cause the robot arm 3 to perform a position movement, rotation, cutting operation, etc.

In one example, the handles 10 can include a main handle and a sub-handle. The operator can manipulate the slave robot arm 3 or the laparoscope 5, etc., with only the main handle, or also manipulate the sub-handle to manipulate multiple surgical equipment simultaneously in real time. The main handle and sub-handle can have various mechanical compositions according to the manipulation method, and various inputting elements can be used, such as a joystick, a keypad, a trackball, a touchscreen, etc., for example, to operate the robot arm 3 of the slave robot 2 and/or other surgical equipment.

The master controller is not limited to the shape of a handle 10, and any type can be applied if it is able to control the operation of a robot arm 3 over a network.

On the monitor unit 6 of the master interface 4, a picture inputted by the laparoscope 5 may be displayed as an on-screen image. A virtual surgical tool controlled by the operator manipulating the handles 10 can also be displayed together on the monitor unit 6 or on an independent screen. Furthermore, the information displayed on the monitor unit 6 can be varied according to the selected drive mode. The displaying of the virtual surgical tool, its control method, the displayed information for each drive mode, and the like, will be described later in more detail with reference to the relevant drawings.

The monitor unit 6 can be composed of one or more monitors, each of which can individually display information required during surgery. While FIG. 1 and FIG. 2 illustrate an example in which the monitor unit 6 includes three monitors, the number of monitors can be varied according to the type or characteristic of the information that needs to be displayed.

The monitor unit 6 can further output multiple sets of vital information related to the patient. In this case, one or more sets of vital information, such as body temperature, pulse rate, respiratory rate, blood pressure, etc., for example, can be outputted through one or more monitors of the monitor unit 6, where each information can be outputted in a separate area. To provide the master robot 1 with this vital information, the slave robot 2 can include a vital information measurement unit, which may include one or more of a body temperature measurement module, a pulse rate measurement module, a respiratory rate measurement module, a blood pressure measurement module, an electrocardiographic measurement module, etc. The vital information measured by each module can be transmitted from the slave robot 2 to the master robot 1 in the form of analog signals or digital signals, and the master robot 1 can display the received vital information through the monitor unit 6.

The slave robot 2 and the master robot 1 can be interconnected by a wired or a wireless network to exchange with each other manipulation signals, laparoscope pictures inputted through the laparoscope 5, and the like. If there are two manipulation signals originating from the two handles 10 equipped on the master interface 4 and/or a manipulation signal for a position adjustment of the laparoscope 5 that have to be transmitted simultaneously and/or at a similar time, each of the manipulation signals can be transmitted to the slave robot 2 independently of one another. Here, to state that each manipulation signal may be transmitted “independently” means that there is no interference between manipulation signals and that no one manipulation signal affects another signal. Various methods can be used to transmit the multiple manipulation signals independently of one another, such as by transmitting the manipulation signals after adding header information for each manipulation signal during the generating the manipulation signals, transmitting the manipulation signals in the order in which they were generated, or pre-setting a priority order for transmitting the manipulation signals, and the like. It is also possible to fundamentally prevent interference between manipulation signals by having independent transmission paths through which the manipulation signals may be transmitted respectively.

The robot arms 3 of the slave robot 2 can be implemented to have high degrees of freedom. A robot arm 3 can include, for example, a surgical tool that will be inserted in the surgical site of the patient, a yaw driving unit for rotating the surgical tool in a yaw direction according to the operating position, a pitch driving unit for rotating the surgical tool in a pitch direction perpendicular to the rotational driving of the yaw driving unit, a transport driving unit for moving the surgical tool along a lengthwise direction, a rotation driving unit for rotating the surgical tool, and a surgical tool driving unit installed on the end of the surgical tool to incise or cut a surgical lesion. However, the composition of the robot arms 3 is not thus limited, and it is to be appreciated that such an example does not limit the scope of claims of the present invention. The actual control procedures by which the robot arms 3 are rotated, moved, etc., in correspondence to the operator manipulating the handles 10 will not be described here in detail, as they are not directly connected with the essence of the invention.

One or more slave robots 2 can be used to perform surgery on a patient, and the laparoscope 5 for displaying the surgical site on the monitor unit 6 as an on-screen image can be implemented on an independent slave robot 2. Also, as described above, the embodiments of the invention can be generally used for surgical operations that employ various surgical endoscopes (e.g. a thoracoscope, arthroscope, rhinoscope, etc.), other than a laparoscope.

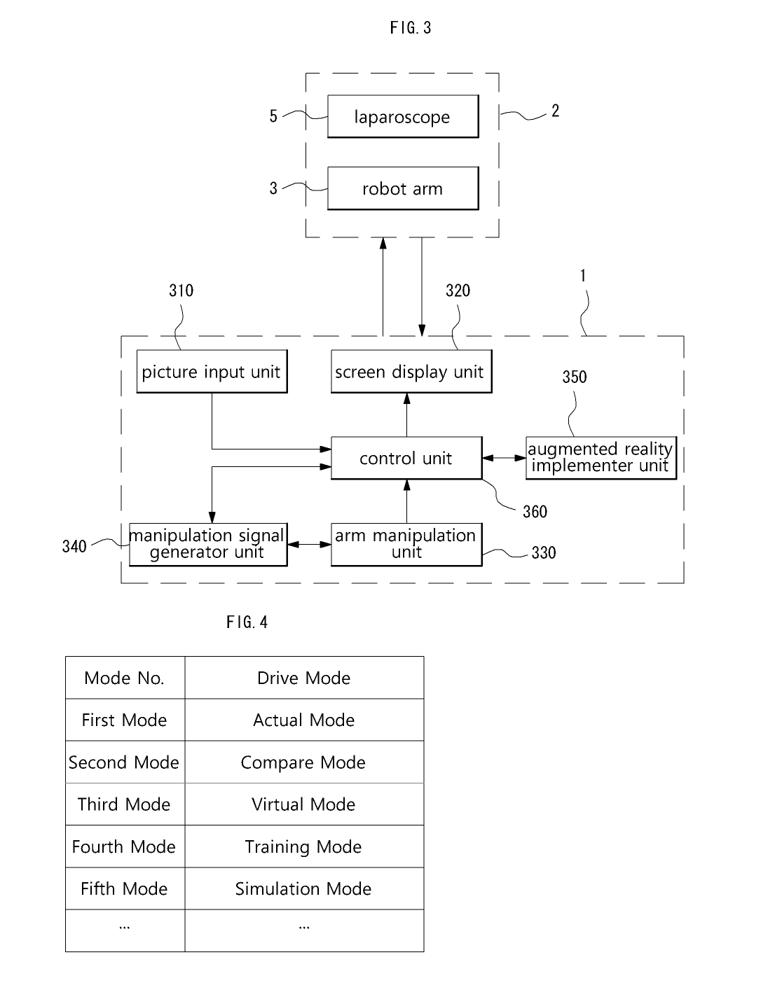

FIG. 3 is a block diagram schematically illustrating the composition of a master robot and a slave robot according to an embodiment of the invention, while FIG. 4 illustrates an example of drive modes for a surgical robot system according to an embodiment of the invention, and FIG. 5 illustrates an example of a mode indicator showing an active drive mode according to an embodiment of the invention.

Referring to FIG. 3, which schematically depicts the compositions of the master robot 1 and the slave robot 2, the master robot 1 may include a picture input unit 310, a screen display unit 320, an arm manipulation unit 330, a manipulation signal generator unit 340, an augmented reality implementer unit 350, and a control unit 360. The slave robot 2 may include a robot arm 3 and a laparoscope 5. While it is not illustrated in FIG. 3, the slave robot 2 can further include a vital information measurement unit, etc., for measuring and providing vital information related to the patient. Also, the master robot 1 can further include a speaker unit for outputting warning information, such as a warning sound, a warning voice message, etc., when it is determined that an emergency situation has occurred.

The picture input unit 310 may receive, over a wired or a wireless network, a picture inputted through a camera equipped on the laparoscope 5 of the slave robot 2.

The screen display unit 320 may output an on-screen image, which corresponds to a picture received through the picture input unit 310, as visual information. Also, the screen display unit 320 can further output a virtual surgical tool as visual information according to the manipulation on the arm manipulation unit 330, and if vital information is inputted from the slave robot 2, can also output information corresponding to the vital information. The screen display unit 320 can be implemented in the form of a monitor unit 6, etc., and a picture processing process for outputting the received picture through the screen display unit 320 as an on-screen image can be performed by the control unit 360, the augmented reality implementer unit 350, or by a picture processing unit (not shown).

The arm manipulation unit 330 may enable the operator to manipulate the position and function of the robot arm 3 of the slave robot 2. Although the arm manipulation unit 330 can be formed in the shape of a handle 10, as illustrated in FIG. 2, the shape is not thus limited and can be implemented in a variety of shapes as long as the same purpose is achieved. Furthermore, in certain examples, a portion can be formed in the shape of a handle, while another portion can be formed in a different shape, such as a clutch button, etc., and finger insertion tubes or insertion rings can be formed that are inserted and secured onto the operator's fingers to facilitate the manipulation of the surgical tools.

As described above, the arm manipulation unit 330 can be equipped with a clutch button 14, and the clutch button 14 can also be used as a mode-changing control button. Alternatively, the mode-changing control button can be implemented in a mechanical form such as a pedal (not shown), etc., or can also be implemented as a function menu or a selection menu, etc. If the laparoscope 5 from which pictures may be inputted is such that can have its position and/or picture-inputting angle moved or changed by a control of the operator, instead of being fixed in a particular position, then the clutch button 14, etc., can also be configured for adjusting the position and/or picture-inputting angle of the laparoscope 5.

When an operator manipulates an arm manipulation unit 330 in order to achieve a position movement or a maneuver for a surgical action for the robot arm 3 and/or the laparoscope 5, the manipulation signal generator unit 340 may generate and transmit a corresponding manipulation signal to the slave robot 2. The manipulation signal can be transmitted and received over a wired or wireless communication, as already described above.

The augmented reality implementer unit 350 may provide the processing that enables the screen display unit 320 to display not only the picture of the surgical site, which is inputted through the laparoscope 5, but also the virtual surgical tool, which moves in conjunction with manipulations on the arm manipulation unit 330 in real time, when the master robot 1 is driven in the second mode, i.e. the compare mode, etc. The specific functions and various details, etc., of the augmented reality implementer unit 350 are described later in more detail with reference to the relevant drawings.

The control unit 360 may control the actions of each of the component parts so that the functions described above may be implemented. The control unit 360 can also serve to convert a picture inputted through the picture input unit 310 into an on-screen image that will be displayed through the screen display unit 320. Also, if manipulation information is inputted according to a manipulation on the arm manipulation unit 330, the control unit 360 may control the augmented reality implementer unit 350 correspondingly such that the virtual surgical tool is outputted through the screen display unit 320. The control unit 360 can also provide control to endow or retrieve surgery authority in the fourth mode, i.e. the training mode, between a learner and a trainer.

As in the example shown in FIG. 4, the master robot 1 and/or slave robot 2 can be operated in a drive mode selected by the operator, etc., from among various drive modes.

For example, the drive mode can include a first mode of actual mode, a second mode of compare mode, a third mode of virtual mode, a fourth mode of training mode, a fifth mode of simulation mode, and so on.

When the master robot 1 and/or slave robot 2 operate in the first mode, i.e. the actual mode, the picture displayed through the monitor unit 6 of the master robot 1 can include the surgical site, the actual surgical tool, etc., as in the example shown in FIG. 5. In other words, the display can exclude the virtual surgical tool, to be identical or similar to the display screen shown during remote surgery using a conventional surgical robot system. Of course, even when operating in the first mode, if the patient's vital information is measured by the slave robot 2 and received, the corresponding information can be displayed, and as already described above, various methods can be used for displaying this information.

When the master robot 1 and/or slave robot 2 operate in the second mode, i.e. the compare mode, the picture displayed through the monitor unit 6 of the master robot 1 can include the surgical site, the actual surgical tool, the virtual surgical tool, etc.

The actual surgical tool, as used herein, refers to a surgical tool that is included in the picture that is inputted by the laparoscope 5 and transmitted to the master robot 1, and is the surgical tool that directly applies a surgical action on the patient's body. In contrast, the virtual surgical tool is controlled by the manipulation information (i.e. the information related to the movement, rotation, etc., of a surgical tool) recognized by the master robot 1 as the operator manipulates the arm manipulation unit 330 and is a surgical tool that is displayed virtually only on the screen. The positions and manipulation shapes of the actual surgical tool and the virtual surgical tool would be decided by the manipulation information.

The manipulation signal generator unit 340 may generate a manipulation signal, using the manipulation information resulting from the operator's manipulation on the arm manipulation unit 330, and may transmit the generated manipulation signal to the slave robot 2, so that consequently the actual surgical tool may be manipulated in correspondence with the manipulation information. Moreover, the position and manipulation shape of the actual surgical tool manipulated by the manipulation signal can be checked by the operator from the picture inputted by the laparoscope 5. That is, if the network communication speed between the master robot 1 and the slave robot 2 is sufficiently fast, then the actual surgical tool and the virtual surgical tool would move at similar speeds. In contrast, if the network communication speed is somewhat slow, then the virtual surgical tool would move first and the actual surgical tool would move in a manner identical to the manipulation of the virtual surgical tool, after a slight interval in time. However, if the network communication speed is slow (e.g. with a delay exceeding 150 ms), then the actual surgical tool would move after the virtual surgical tool with a certain interval in time.

When the master robot 1 and/or slave robot 2 operate in the third mode, i.e. the virtual mode, the manipulation signal from a learner (i.e. a training student) or a trainer (i.e. a training instructor) on the arm manipulation unit 330 can be made not to be transmitted by the master robot 1 to the slave robot 2, while the picture displayed through the monitor unit 6 of the master robot 1 can include one or more of the surgical site and the virtual surgical tool, etc. The trainer, etc., can select the third mode and perform a preliminary test operation of the actual surgical tool. It can be provided such that entering the third mode is achieved by selecting a clutch button 14, etc., so that while the corresponding button is pressed (or while the third mode is selected), manipulating the handles 10 does not cause the actual surgical tool to move but causes only the virtual surgical tool to move. Also, when entering the third mode, or the virtual mode, the settings can be made such that only the virtual surgical tool moves unless there is a special manipulation by the trainer, etc. While in this state, when the pressing of the corresponding button is stopped (or the first mode or second mode is selected), or the virtual mode is stopped, the actual surgical tool can be moved to conform with the manipulation information by which the virtual surgical tool was moved, or the handles 10 can be restored (or the position and manipulation form of the virtual surgical tool can be restored) to the time point at which the corresponding button was pressed.

When the master robot 1 and/or slave robot 2 operate in the fourth mode, i.e. the training mode, the manipulation signal from the learner (i.e. the training student) or the trainer (i.e. the training instructor) on the manipulation unit 330 can be transmitted to the master robot 1 that is manipulated by the trainer or the learner. To this end, one slave robot 2 can be connected with two or more master robots 1, or the master robot 1 can be connected with another separate master robot 1. In this case, when the arm manipulation unit 330 of a trainer's master robot 1 is manipulated, the corresponding manipulation signal can be transferred to the slave robot 2, and the picture inputted through the laparoscope 5 can be displayed through the monitor unit 6 of each of the trainer's and the learner's master robots 1 to check surgery progress. On the other hand, when the arm manipulation unit 330 of the learner's master robot 1 is manipulated, the corresponding manipulation signal can be provided only to the trainer's master robot 1 and not to the slave robot 2. Thus, the trainer's manipulation can function as in the first mode, while the learner's manipulation can function as in the third mode. Operations in the fourth mode, or the training mode, will be described later in more detain with reference to the related drawings.

When operating in the fifth mode, i.e. the simulation mode, the master robot 1 may serve as a surgery simulator that uses the characteristics (e.g. shape, texture, tactile feel during incision, etc.) of an organ shaped in 3 dimensions by 3-dimensional modeling. That is, the fifth mode can be understood as being similar to the third mode, or the virtual mode, but more advanced, as the function of a surgery simulator can be provided in which the characteristics of an organ can be coupled with a 3-dimensional shape obtained by using a stereo endoscope, etc.

If the liver is outputted through the screen display unit 320, a stereo endoscope can be used to identify the shape of the liver, which can be matched with mathematically modeled characteristic information of the liver (this information can be stored beforehand in a storage unit (not shown)), to enable surgery simulation during surgery in virtual mode. For example, one may perform a surgery simulation, with the characteristic information of the liver matched with the shape of the liver, to see which is the proper direction in which to excise the liver, before actually excising the liver. Furthermore, based on the mathematical modeling information and the characteristic information, one can experience the tactile feel provided during surgery, to see which portion is hard and which portion is soft. In this case, an organ's surface shape information, which may be obtained 3-dimensionally, can be aligned with a 3-dimensional shape of the organ's surface reconstructed by referencing a CT (computer tomography) or/and MRI (magnetic resonance image) picture, etc., while a 3-dimensional shape of the organ's interior reconstructed from a CT, MRI picture, etc., can be aligned with mathematically modeled information, to enable a more realistic surgery simulation.

The third mode (virtual mode) and/or the fifth mode (simulation mode) described above can also be employed in applying a method of performing surgery using history information, which will be described later in more detail with reference to the related drawings.

While a description has been provided above of drive modes ranging from the first mode to the fifth mode, it is also possible to add other drive modes for various purposes.

Also, when the master robot 1 is driven in each mode, it can be confusing for the operator to know which mode the drive mode is currently in. To enable accurate distinguishing between drive modes, the screen display unit 320 can further display a mode indicator.

FIG. 5 shows an example of how a mode indicator can be further displayed on a screen displaying the surgical site and the actual surgical tool 460. The mode indicator enables clear recognition of the current drive mode and can be of various forms, such as, for example, a message 450, a boundary color 480, etc. Besides this, the mode indicator can also be implemented as an icon, a background color, etc., and it is possible to display just a single mode indicator or display two or more mode indicators together.

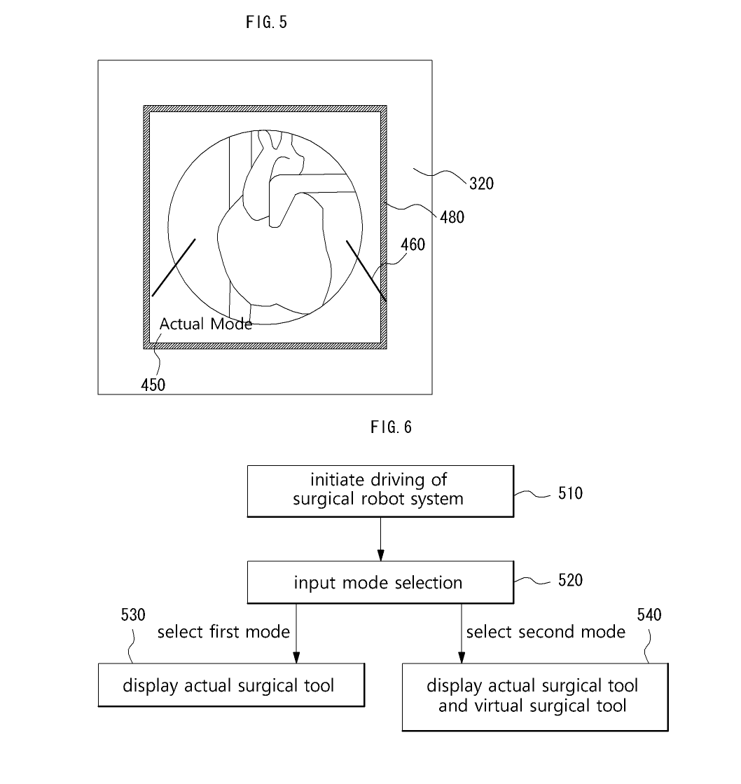

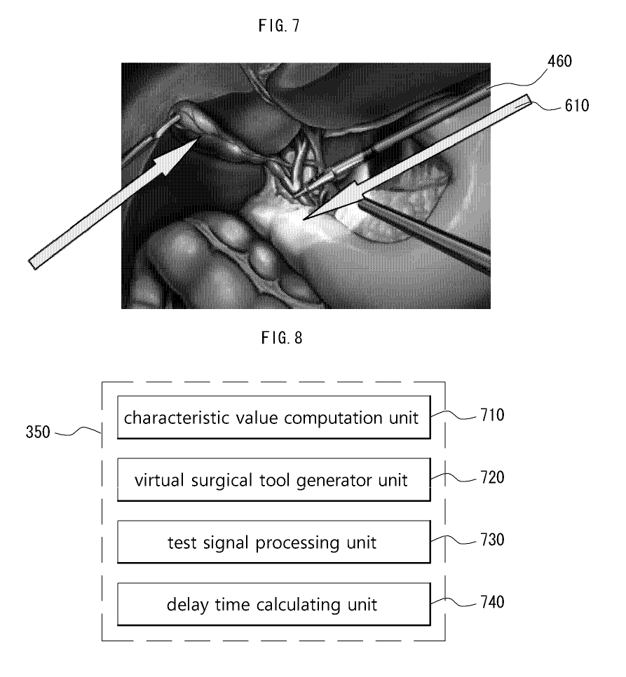

FIG. 6 is a flowchart illustrating a procedure of selecting a drive mode between a first mode and a second mode, and FIG. 7 illustrates an example of a screen display outputted through a monitor unit in the second mode according to an embodiment of the invention.

While FIG. 6 shows an example in which it is assumed that either the first mode or the second mode is selected, if the drive modes are applied in a first mode through a fifth mode as in the example shown in FIG. 4, the mode selection input in step 520 described below can be for one of the first mode through the fifth mode, and in step 530 and step 540, the screen display can be performed according to the mode selected.

Referring to FIG. 6, the driving of the surgical robot system may be initiated in step 510. After initiating the driving of the surgical robot system, the picture inputted through the laparoscope 5 would be outputted through the monitor unit 6 of a master robot 1.

In step 520, the master robot 1 may receive a selection of a drive mode as input from the operator. The selection of the drive mode can be achieved, for example, by pressing a mechanically implemented clutch button 14 or pedal (not shown), or by using a function menu or mode selection menu, etc., displayed through the monitor unit 6.

If the first mode is selected in step 520, the master robot 1 may operate in the drive mode of actual mode, and may display on the monitor unit 6 a picture inputted from the laparoscope 5.

However, if the second mode is selected in step 520, the master robot 1 may operate in the drive mode of compare mode, and may display on the monitor unit 6 not only the picture inputted from the laparoscope 5, but also the virtual surgical tool that is controlled by manipulation information according to manipulations on the arm manipulation unit 330.

FIG. 7 shows an example of a screen display that may be outputted through the monitor unit 6 in the second mode.

As in the example shown in FIG. 7, in the compare mode, a picture inputted and provided by the laparoscope 5 (i.e. a picture displaying the surgical site and the actual surgical tool 460), as well as the virtual surgical tool 610 controlled by the manipulation information according to the arm manipulation unit 330 may be displayed together on the screen.

The difference in display position, etc., between the actual surgical tool 460 and the virtual surgical tool 610 can be caused by the network communication speed between the master robot 1 and the slave robot 2, and after a period of time, the actual surgical tool 460 would be displayed moved to the current position of the current virtual surgical tool 610.

While FIG. 7 shows an example in which the virtual surgical tool 610 is represented in the shape of an arrow for purposes of differentiation from the actual surgical tool 460, the display shape of the virtual surgical tool 610 can be processed to be identical to the display shape of the actual surgical tool, or can be represented in various forms for easier differentiation, such as a translucent form, a dotted outline, etc. Details regarding whether or not to display the virtual surgical tool 610 and in what form will be provided later on with reference to the related drawings.

Also, various methods can be used for displaying the picture inputted and provided by the laparoscope 5 together with the virtual surgical tool 610, such as by displaying the virtual surgical tool 610 to be superimposed over the laparoscope picture, and by reconstructing the laparoscope picture and the virtual surgical tool 610 as a single picture, for example.

FIG. 8 illustrates the detailed composition of an augmented reality implementer unit 350 according to an embodiment of the invention, and FIG. 9 is a flowchart illustrating a method of driving a master robot 1 in the second mode according to an embodiment of the invention.

Referring to FIG. 8, the augmented reality implementer unit 350 can include a characteristic value computation unit 710, a virtual surgical tool generator unit 720, a test signal processing unit 730, and a delay time calculating unit 740. Some of the components (e.g. the test signal processing unit 730, delay time calculating unit 740, etc.) of the augmented reality implementer unit 350 can be omitted, while some components (e.g. a component for processing the vital information received from the slave robot 2 such that it can be outputted through the screen display unit 320, and the like) can be added. One or more of the components included in the augmented reality implementer unit 350 can also be implemented in the form of a software program composed of a combination of program codes.

The characteristic value computation unit 710 may compute characteristic values by using the picture inputted and provided by the laparoscope 5 of the slave robot 2 and/or coordinate information regarding the position of the actual surgical tool coupled to the robot arm 3. The position of the actual surgical tool can be recognized by referencing the position value of the robot arm 3 of the slave robot 2, and the information related to the corresponding position can also be provided to the master robot 1 from the slave robot 2.

The characteristic value computation unit 710 can compute characteristic values such as the laparoscope's 5 field of view (FOV), magnifying ratio, viewpoint (e.g. viewing direction), and viewing depth, and the actual surgical tool's 460 type, direction, depth, and degree of bending, and so on, for example, by using the picture from the laparoscope 5, etc. In cases where the characteristic values are computed using the picture from the laparoscope 5, image-recognition technology can be employed, for extracting the contours of an object included in the picture, recognizing its shape, recognizing its inclination angle, etc. Also, the type, etc., of the actual surgical tool 460 can be inputted beforehand during the process of coupling the surgical tool to the robot arm 3.

The virtual surgical tool generator unit 720 may generate the virtual surgical tool 610 that is to be displayed through the screen display unit 320, by referencing the manipulation information resulting from the operator's manipulation of the robot arm 3. The position at which the virtual surgical tool 610 is initially displayed can be based, for example, on the display position at which the actual surgical tool 460 is displayed through the screen display unit 320, and the movement displacement of the virtual surgical tool 610 manipulated according to the manipulation on the arm manipulation unit 330 can, for example, be set beforehand by referencing measured values by which the actual surgical tool 460 moves in correspondence with the manipulation signals.

The virtual surgical tool generator unit 720 can also generate only the virtual surgical tool information (e.g. the characteristic values for expressing the virtual surgical tool) for outputting the virtual surgical tool 610 through the screen display unit 320. In deciding the shape or position of the virtual surgical tool 610 according to the manipulation information, the virtual surgical tool generator unit 720 can also reference the characteristic values computed by the characteristic value computation unit 710 or the characteristic values used immediately before for expressing the virtual surgical tool 610. This can allow a prompt generation of the corresponding information, for cases in which only a translational movement is made, with the virtual surgical tool 610 or the actual surgical tool 460 maintaining the same arrangement (e.g. inclination angle, etc.) as before.

The test signal processing unit 730 may transmit a test signal to the slave robot 2 and may receive a response signal from the slave robot 2, in order to determine the network communication speed between the master robot 1 and the slave robot 2. The test signal transmitted by the test signal processing unit 730 can be, for example, a typical signal used incorporated in the form of a time stamp in a control signal exchanged between the master robot 1 and the slave robot 2, or can be a signal used additionally for measuring the network communication speed. Also, certain time points, from among all of the time points at which the test signal is exchanged, can be pre-designated as time points at which the network communication speed measurement is performed.

The delay time calculating unit 740 may calculate the delay time of the network communication by using the transmission time of the test signal and the reception time of the response signal. If the network communication speed is the same between the segment at which the master robot 1 transmits a certain signal to the slave robot 2 and the segment at which the master robot 1 receives a certain signal from the slave robot 2, then the delay time can be, for example, ½ of the difference between the transmission time of the test signal and the reception time of the response signal. This is because the slave robot would immediately perform a corresponding processing upon receiving a manipulation signal from the master robot 1. Of course, the delay time can also additionally include a processing delay time at the slave robot 2 for performing a processing, such as controlling the robot arm 3 according to the manipulation signals. In another example, if the difference between the operator's manipulating time and observing time is of importance, the delay time can also be calculated as the difference between the transmission time and the reception time of the response signal (e.g. the time at which the operator's manipulation result is displayed through the display unit). Various other approaches can be used for calculating the delay time, other than those described above.

If the delay time is equal to or shorter than a pre-designated threshold value (e.g. 150 ms), then the difference in display position, etc., between the actual surgical tool 460 and the virtual surgical tool 610 would not be great. In this case, the virtual surgical tool generator unit 720 can make it so that the virtual surgical tool 610 is not displayed through the screen display unit 320. This is because it is not necessary to doubly display the actual surgical tool 460 and the virtual surgical tool 610 at agreeing or proximate positions and cause confusion for the operator.

However, if the delay time exceeds a pre-designated threshold value (e.g. 150 ms), then the difference in display position, etc., between the actual surgical tool 460 and the virtual surgical tool 610 can be great. In this case, the virtual surgical tool generator unit 720 can make it so that the virtual surgical tool 610 is displayed through the screen display unit 320. This is to eliminate possible confusion for the operator caused by a real time disagreement between the manipulation on the operator's arm manipulation unit 330 and the manipulation of the actual surgical tool 460. Thus, even if the operator performs surgery by referencing the virtual surgical tool 610, the actual surgical tool 460 will be subsequently manipulated in the same manner as the manipulation of the virtual surgical tool 610.

FIG. 9 shows a flowchart for an example of a method of driving a master robot 1 in the second mode. In describing each step of the flowchart, it will be assumed for convenience both in explanation and comprehension, that the master robot 1 performs each step.

Referring to FIG. 9, in step 810, the master robot 1 may generate a test signal for measuring network communication speed and transmit the test signal to the slave robot 2 over a wired or a wireless network.

In step 820, the master robot 1 may receive a response signal from the slave robot 2 in response to the test signal.

In step 830, the master robot 1 may calculate the delay time for the network communication speed by using the transmission time of the test signal and the reception time of the response signal.

Then, in step 840, the master robot 1 may determine whether or not the calculated delay time is equal to or shorter than a preset threshold value. Here, the threshold value may be the delay time for the network communication speed that is required by the operator to adequately perform surgery using the surgical robot system, and can be applied after it is decided using an empirical and/or statistical method.

If the calculated delay time is equal to or shorter than the preset threshold value, the process proceeds to step 850, in which the master robot 1 may provide processing such that a picture inputted via the laparoscope 5 (i.e. a picture including the surgical site and the actual surgical tool 460) is displayed on the screen display unit 320. Here, the virtual surgical tool 610 can be excluded from being displayed. Of course, in this case also, it is possible to have both the virtual surgical tool 610 and the actual surgical tool 460 displayed together.

However, if the calculated delay time exceeds the preset threshold value, the process proceeds to step 860, in which the master robot 1 may provide processing such that the picture inputted via the laparoscope 5 (i.e. a picture including the surgical site and the actual surgical tool 460) and the virtual surgical tool 610 are displayed together on the screen display unit 320. Of course, in this case also, it is possible to have the virtual surgical tool 610 excluded from being displayed.

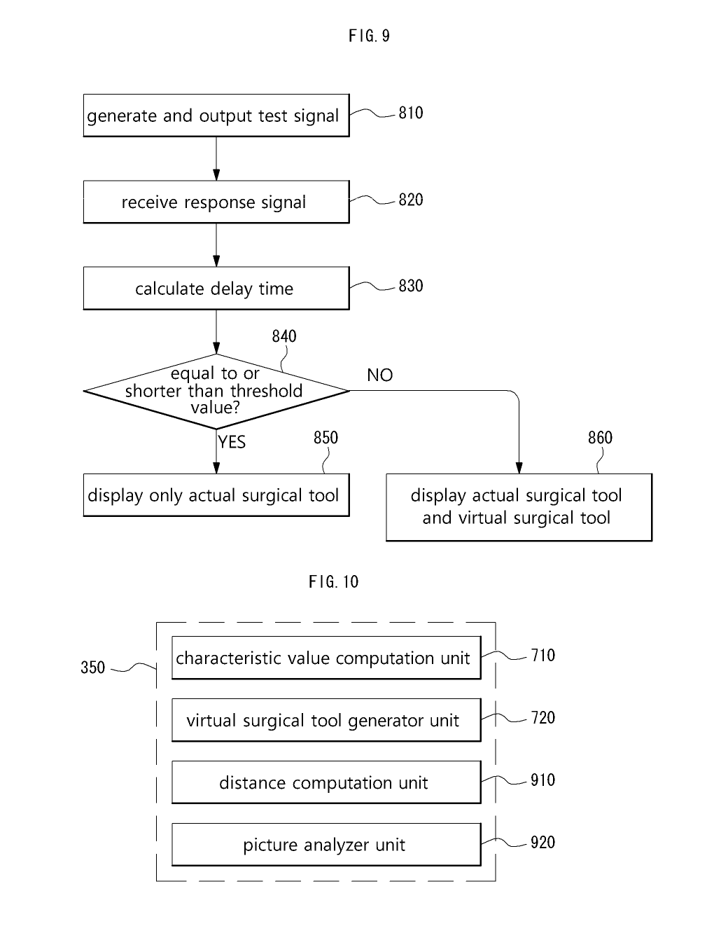

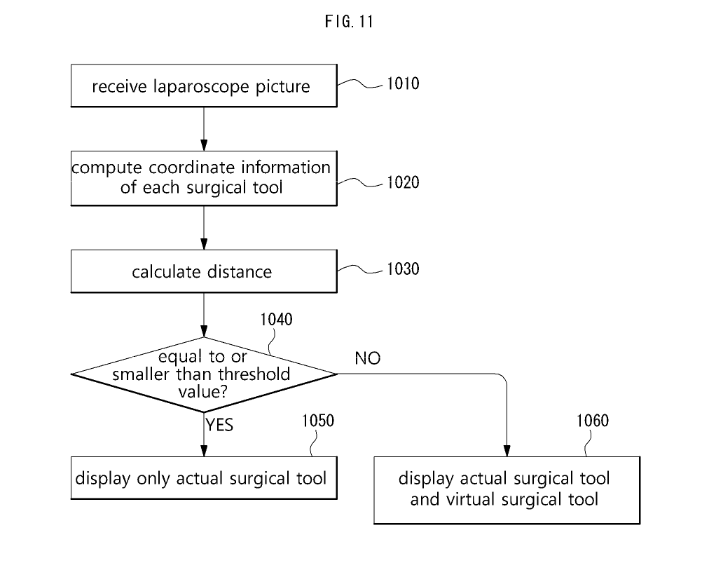

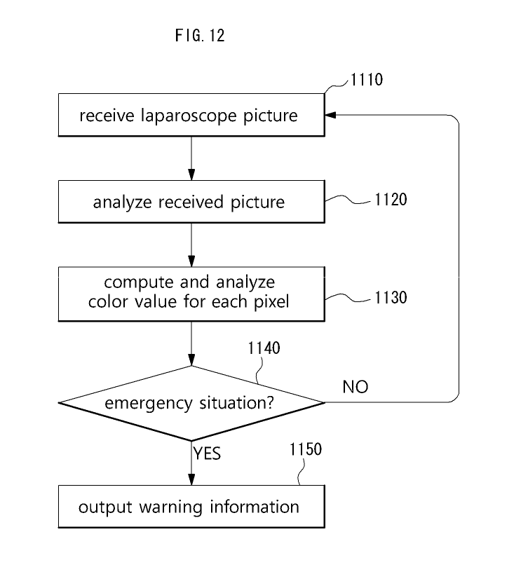

FIG. 10 illustrates the detailed composition of an augmented reality implementer unit 350 according to another embodiment of the invention, while FIG. 11 and FIG. 12 are flowcharts respectively illustrating methods of driving a master robot 1 in the second mode according to different embodiments of the invention.

Referring to FIG. 10, the augmented reality implementer unit 350 may include a characteristic value computation unit 710, a virtual surgical tool generator unit 720, a distance computation unit 910, and a picture analyzer unit 920. Some of the components of the augmented reality implementer unit 350 can be omitted, while some components (e.g. a component for processing the vital information received from the slave robot 2 such that it can be outputted through the screen display unit 320, and the like) can be added. One or more of the components included in the augmented reality implementer unit 350 can also be implemented in the form of a software program composed of a combination of program codes.

The characteristic value computation unit 710 may compute the characteristic values by using the picture inputted and provided by the laparoscope 5 of the slave robot 2 and/or coordinate information regarding the position of the actual surgical tool coupled to the robot arm 3. The characteristic values can include, for example, one or more of the laparoscope's 5 field of view (FOV), magnifying ratio, viewpoint (e.g. viewing direction), viewing depth, etc., and the actual surgical tool's 460 type, direction, depth, degree of bending, etc.

The virtual surgical tool generator unit 720 may generate the virtual surgical tool 610 that is to be displayed through the screen display unit 320, by referencing the manipulation information resulting from the operator's manipulation of the robot arm 3.

The distance computation unit 910 may use the position coordinates of the actual surgical tool 460 computed by the characteristic value computation unit 710 and the position coordinates of the virtual surgical tool 610 that moves in conjunction with manipulations on the arm manipulation unit 330, to compute the distance between the surgical tools. For example, when the position coordinates of the virtual surgical tool 610 and the actual surgical tool 460 are decided, the length of the line segment connecting the two points can be computed. Here, the position coordinates can be, for example, the coordinate values of a point in 3-dimensional space defined by the x-y-z axes, and a corresponding point can be pre-designated to be a point at a particular position on the virtual surgical tool 610 and the actual surgical tool 460. In addition, obtaining the distance between the surgical tools can also utilize the length of a path or a trajectory generated by the manipulation method. This is because, if a circle is drawn, for example, and a delay exists during the drawing of the circle, then the length of the line segment between the surgical tools may be very small, although the length of the path or trajectory may be as long as the circumference of the circle generated by the manipulation method.

The position coordinates of the actual surgical tool 460, used for computing distance, can be applied as absolute coordinate values or relative coordinate values with respect to a particular point, or the position of the actual surgical tool 460 as displayed through the screen display unit 320 can be coordinatized. Similarly, for the position coordinates of the virtual surgical tool 610 also, the virtual position moved by manipulations on the arm manipulation unit 330 can be applied as absolute coordinates with respect to the initial position of the virtual surgical tool 610, or relative coordinate values computed with respect to a particular point can be used, or the position of the virtual surgical tool 610 as displayed through the screen display unit 320 can be coordinatized. Here, analyzing the position of each surgical tool displayed through the screen display unit 320 can employ feature information obtained by the picture analyzer unit 920 described below.

If the distance between the virtual surgical tool 610 and the actual surgical tool 460 is small or is 0, then the network communication speed can be considered to be adequate, but if the distance is large, then the network communication speed can be considered to be insufficient.

Using the distance information computed by the distance computation unit 910, the virtual surgical tool generator unit 720 can decide on one or more issues of whether or not to display the virtual surgical tool 610, and the color, form, etc., in which the virtual surgical tool 610 is to be displayed in. For example, if the distance between the virtual surgical tool 610 and the actual surgical tool 460 is equal to or smaller than a preset threshold value, it can be made such that the virtual surgical tool 610 is not outputted through the screen display unit 320. Also, if the distance between the virtual surgical tool 610 and the actual surgical tool 460 exceeds the preset threshold value, a processing can be provided such that the operator has a clear recognition of the network communication speed, for example, by adjusting the translucency, distorting the color, or changing the contour thickness of the virtual surgical tool 610, in proportion to the distance. Here, the threshold value can be designated to be a distance value, such as 5 mm, etc., for example.

The picture analyzer unit 920 may extract preset feature information (e.g. one or more of a color value for each pixel, and the actual surgical tool's position coordinates, manipulation shape, etc.) by using the picture inputted and provided by the laparoscope 5. For example, in order to allow immediate countermeasures in the event of an emergency situation (e.g. excessive bleeding, etc.) during surgery, the picture analyzer unit 920 can analyze the color value for each pixel of the corresponding picture to determine whether or not the pixels having a color value representing blood exceed a base value, or determine whether or not an area or region formed by the pixels having a color value representing blood is equal to or greater than a particular size. Also, the picture analyzer unit 920 can capture the display screen of the screen display unit 320, on which the picture inputted by the laparoscope 5 and the virtual surgical tool 610 are displayed, to generate the position coordinates of the respective surgical tools.

FIG. 11 is a flowchart illustrating a method of driving the master robot 1 in the second mode according to another embodiment of the invention.

Referring to FIG. 11, in step 1010, the master robot 1 may receive a laparoscope picture (i.e. the picture inputted and provided through the laparoscope 5) from the slave robot 2.

In step 1020, the master robot 1 may compute the coordinate information of the actual surgical tool 460 and the virtual surgical tool 610. Here, the coordinate information can be computed, for example, by using the characteristic values computed by the characteristic value computation unit 710 and the manipulation information, or by using feature information extracted by the picture analyzer unit 920.

In step 1030, the master robot 1 may compute the distance between the surgical tools, by using the coordinate information computed for each surgical tool.

In step 1040, the master robot 1 may determine whether or not the computed distance is equal to or smaller than a threshold value.

If the computed distance is equal to or smaller than a threshold value, the process may proceed to step 1050, in which the master robot 1 may output the laparoscope picture through the screen display unit 320 but not display the virtual surgical tool 610.

However, if the computed distance exceeds the threshold value, the process may proceed to step 1060, in which the master robot 1 may display the laparoscope picture and the virtual surgical tool 610 together through the screen display unit. Here, a processing can be provided such as of adjusting the translucency, distorting the color, or changing the contour thickness of the virtual surgical tool 610, in proportion to the distance.

Also, FIG. 12 is a flowchart illustrating a method of driving the master robot 1 in the second mode according to yet another embodiment of the invention.

Referring to FIG. 12, in step 1110, the master robot 1 may receive a laparoscope picture. The received laparoscope picture would be outputted through the screen display unit 320.

In step 1120 and step 1130, the master robot 1 may analyze the received laparoscope picture, to compute and evaluate the color value for each pixel of the corresponding picture. Computing the color value for each pixel can be performed by the picture analyzer unit 920, as in the example described above, or by the characteristic value computation unit 710 to which picture recognition technology has been applied. Also, the evaluation of the color value for each pixel can be used to compute one or more of color value frequency, and an area or region formed by pixels having a color value targeted for evaluation, etc.

In step 1140, the master robot 1 may determine whether or not there is an emergency situation, based on the information evaluated in step 1130. The types of emergency situations (e.g. excessive bleeding, etc.) or a basis for determining how the evaluated information should be to be perceived as an emergency situation, and so on, can be defined beforehand.

If it is determined that an emergency situation has occurred, the process may proceed to step 1150, in which the master robot 1 may output warning information. The warning information can be, for example, a warning message outputted through the screen display unit, a warning sound outputted through a speaker unit (not shown), and the like. While it is not illustrated in FIG. 3, the master robot 1 can obviously further include a speaker unit for outputting the warning information or assistance announcements. If, at the time it is determined that an emergency situation has occurred, the virtual surgical tool 610 is being displayed together through the screen display unit 320, then a control can be provided such that the virtual surgical tool 610 is not displayed, so as to enable the operator to make accurate judgments regarding the surgical site.

However, if it is determined that there is no emergency situation, then the process may again proceed to step 1110.

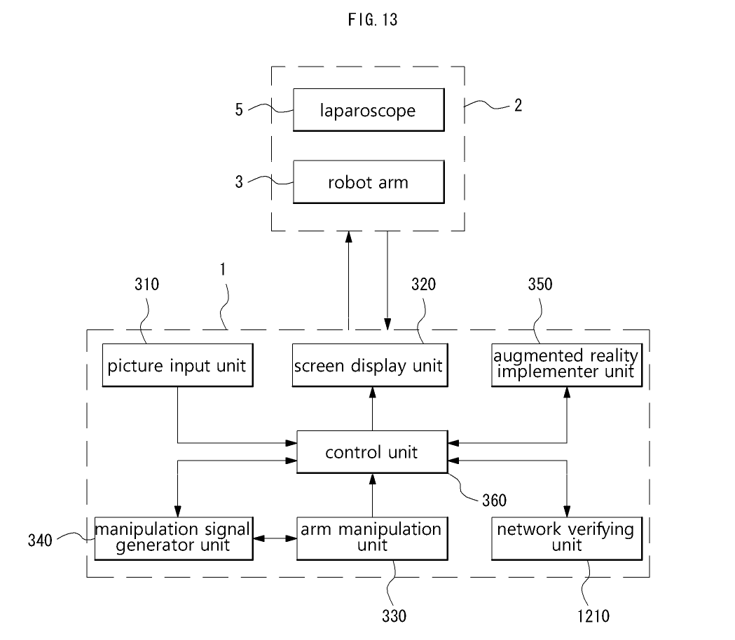

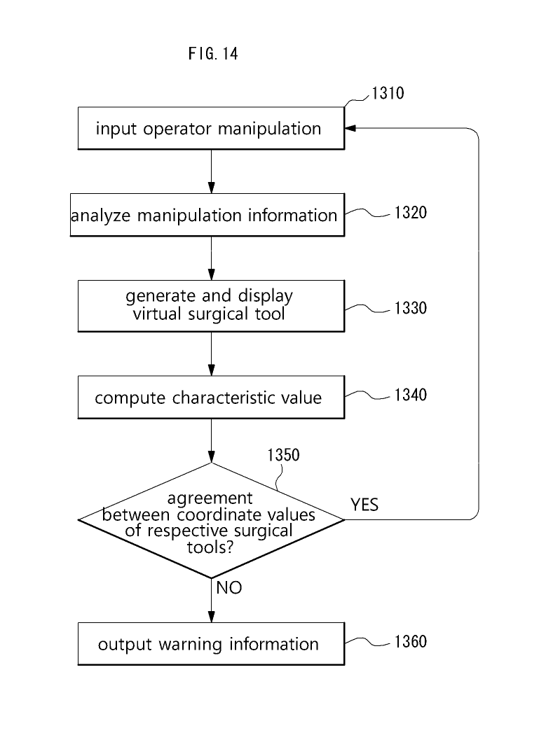

FIG. 13 is a block diagram schematically illustrating the composition of a master robot and a slave robot according to yet another embodiment of the invention, and FIG. 14 is a flowchart illustrating a method of verifying normal driving of a surgical robot system according to yet another embodiment of the invention.

Referring to FIG. 13, which schematically represents the composition of the master robot 1 and the slave robot 2, the master robot 1 may include a picture input unit 310, a screen display unit 320, an arm manipulation unit 330, a manipulation signal generator unit 340, an augmented reality implementer unit 350, a control unit 360, and a network verifying unit 1210. The slave robot 2 may include a robot arm 3 and a laparoscope 5.

The picture input unit 310 may receive, over a wired or a wireless network, a picture inputted through a camera equipped on the laparoscope 5 of the slave robot 2.

The screen display unit 320 may output an on-screen image, which corresponds to a picture received through the picture input unit 310 and/or the virtual surgical tool 610 according to manipulations on the arm manipulation unit 330, as visual information.

The arm manipulation unit 330 may enable the operator to manipulate the position and function of the robot arm 3 of the slave robot 2.

When the operator manipulates the arm manipulation unit 330 in order to move the position of the robot arm 3 and/or the laparoscope 5 or to perform a manipulation for surgery, the manipulation signal generator unit 340 may generate a corresponding manipulation signal and transmit it to the slave robot 2.

The network verifying unit 1210 may verify the network communication between the master robot 1 and the slave robot 2, using the characteristic values computed by the characteristic value computation unit 710 and the virtual surgical tool information generated by the virtual surgical tool generator unit 720. One or more characteristic value of for example, the actual surgical tool's 460 position information, direction, depth, degree of bending, etc., and the virtual surgical tool's 610 position information, direction, depth, degree of bending, etc., according to the virtual surgical tool information can be used for this purpose, and the characteristic values and the virtual surgical tool information can be stored in a storage unit (not shown).

According to an embodiment of the invention, when the manipulation information is generated by the operator's manipulation of the arm manipulation unit 330, the virtual surgical tool 610 may be controlled correspondingly, and also the manipulation signal corresponding to the manipulation information may be transmitted to the slave robot 2 to be used for manipulating the actual surgical tool 460. Also, the position movement, etc., of the actual surgical tool 460 manipulated and controlled by the manipulation signal can be checked through the laparoscope picture. In this case, since the manipulation of the virtual surgical tool 610 occurs within the master robot 1, it will generally occur before the manipulation of the actual surgical tool 460, considering the network communication speed, etc.

Therefore, the network verifying unit 1210 can determine whether or not there is normal network communication, by determining whether or not the actual surgical tool 460 is manipulated identically, or substantially identically within a preset tolerance range, to the movement trajectory or manipulation form, etc., of the virtual surgical tool 610, albeit at a later time. For this purpose, the virtual surgical tool information having characteristic values related to the current position, etc., of the actual surgical tool 460 stored in the storage unit can be utilized. Also, the tolerance range can be set, for example, as a distance value between the sets of coordinate information or a time value until a match is recognized, and so on. The tolerance range can be designated arbitrarily, empirically, or/and statistically.

Also, the network verifying unit 1210 can also perform the verification for network communication by using the feature information analyzed by the picture analyzer unit 920.

The control unit 360 may control the actions of each of the component parts so that the functions described above may be implemented. The control unit 360 can also perform various additional functions, as described in examples for other embodiments.

FIG. 14 shows an example of a method of verifying the network communication to verify whether or not there is normal driving.

Referring to FIG. 14, in steps 1310 and 1320, the master robot 1 may receive as input from the operator a manipulation of the arm manipulation unit 330, and may analyze the manipulation information according to the manipulation of the arm manipulation unit 330. The corresponding manipulation information may include information on the manipulation of the arm manipulation unit 330 for moving the position of the actual surgical tool 460, making an incision in the surgical site, etc., for example.

In step 1330, the master robot 1 may generate virtual surgical tool information by using the analyzed manipulation information, and may output a virtual surgical tool 610 on the screen display unit 320 according to the generated virtual surgical tool information. Here, the generated virtual surgical tool information can be stored in a storage unit (not shown).

In step 1340, the master robot 1 may compute characteristic values for the actual surgical tool 460. Computing the characteristic values can be performed, for example, by the characteristic value computation unit 710 or the picture analyzer unit 920.

In step 1350, the master robot 1 may determine whether or not there is a point of agreement between the coordinate values of the respective surgical tools. If the coordinate information of each surgical tool agrees or agrees within a tolerance range, it can be determined that there is a point of agreement between the coordinate values of the respective surgical tools. Here, the tolerance range can be preset, for example, as a distance value, etc., in 3-dimensional coordinates. As described above, since the results of the operator manipulating the arm manipulation unit 330 would be reflected on the virtual surgical tool 610 before the actual surgical tool 460, step 1350 can be performed by determining whether or not the characteristic values for the actual surgical tool 460 agree with the virtual surgical tool information stored in the storage unit.

If there is no point of agreement between the coordinate values, the process may proceed to step 1360, in which the master robot 1 may output warning information. The warning information can be, for example, a warning message outputted through the screen display unit 320, a warning sound outputted through a speaker unit (not shown), and the like.

However, if there is a point of agreement between the coordinate values, it can be determined that the network communication is normal, and the process may proceed again to step 1310.

Step 1310 through step 1360 described above can be performed in real time during the operator's surgical procedure, or can be performed periodically or at preset time points.

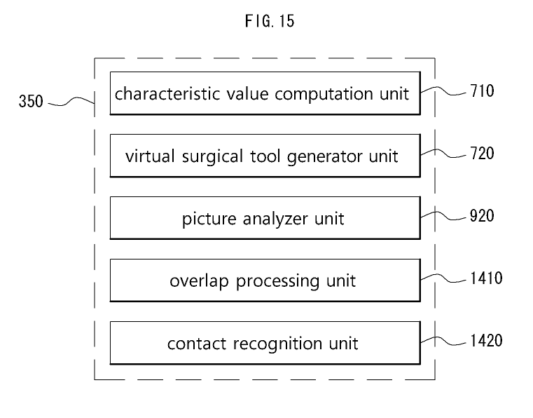

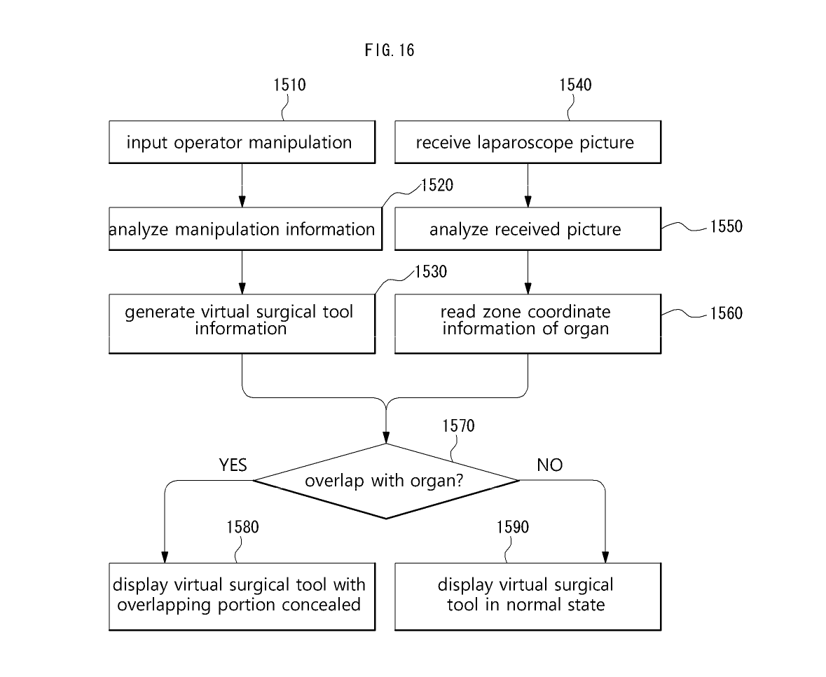

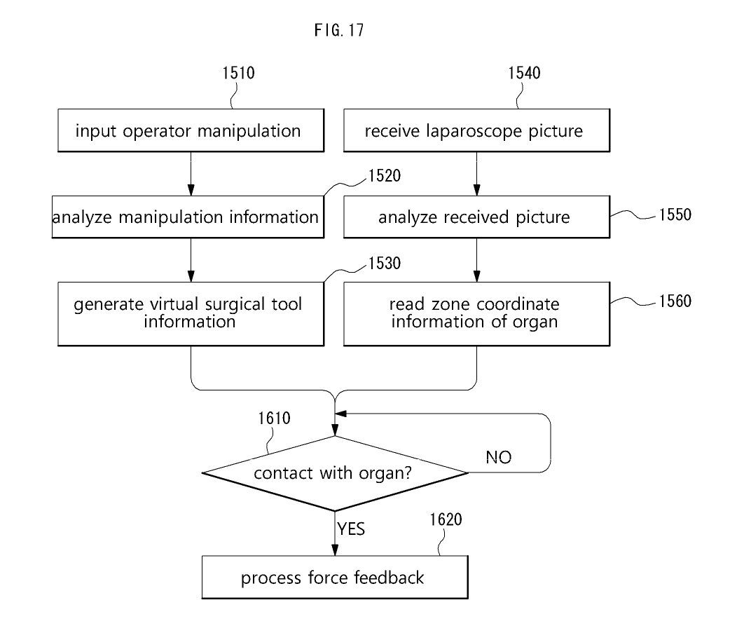

FIG. 15 illustrates the detailed composition of an augmented reality implementer unit 350 according to yet another embodiment of the invention, while FIG. 16 and FIG. 17 are flowcharts respectively illustrating methods of driving a master robot 1 for outputting a virtual surgical tool according to different embodiments of the invention.

Referring to FIG. 15, the augmented reality implementer unit 350 may include a characteristic value computation unit 710, a virtual surgical tool generator unit 720, a picture analyzer unit 920, an overlap processing unit 1410, and a contact recognition unit 1420. Some of the components of the augmented reality implementer unit 350 can be omitted, while some components (e.g. a component for processing the vital information received from the slave robot 2 such that it can be outputted through the screen display unit 320, and the like) can be added. One or more of the components included in the augmented reality implementer unit 350 can also be implemented in the form of a software program composed of a combination of program codes.

The characteristic value computation unit 710 may compute characteristic values by using the picture inputted and provided by the laparoscope 5 of the slave robot 2 and/or coordinate information regarding the position of the actual surgical tool coupled to the robot arm 3. The characteristic values can include one or more of the laparoscope's 5 field of view (FOV), magnifying ratio, viewpoint (e.g. viewing direction), and viewing depth, and the actual surgical tool's 460 type, direction, depth, and bent angle, and so on

The virtual surgical tool generator unit 720 may generate the virtual surgical tool information for outputting the virtual surgical tool 610 through the screen display unit 320, by referencing the manipulation information resulting from the operator's manipulation of the robot arm 3.

The picture analyzer unit 920 may extract preset feature information (e.g. one or more of a shape of an organ within the surgical site, and the actual surgical tool's position coordinates, manipulation shape, etc.) by using the picture inputted and provided by the laparoscope 5. For example, the picture analyzer unit 920 can analyze which organ is being displayed, by using picture recognition technology such as of extracting the contours of the organ displayed in the laparoscope picture, analyzing the color value of each of the pixels depicting the organ, and the like. For this purpose, information related to the shape and color of each organ, the coordination information of a zone in which each organ or/and the surgical site is positioned in 3-dimensional space, and the like, can be pre-stored in a storage unit (not shown). Alternatively, the picture analyzer unit 920 can analyze the coordinate information (absolute coordinates or relative coordinates) of a zone occupied by the corresponding organ, by way of picture analysis.

The overlap processing unit 1410 may use the virtual surgical tool information generated by the virtual surgical tool generator unit 720 and zone coordinate information of an organ and/or the surgical site recognized by the picture analyzer unit 920 to determine whether or not there is overlapping, and may provide processing correspondingly. If a portion of or all of the virtual surgical tool is positioned below or behind an organ, then it can be determined that overlapping (i.e. covering) occurs for the corresponding portion, and in order to increase the reality of the display of the virtual surgical tool 610, processing may be provided such that the area of the virtual surgical tool 610 corresponding to the overlapping portion is concealed (i.e. not displayed through the screen display unit 320). A method of processing to conceal the corresponding overlap portion can employ, for example, a method of applying transparency to the overlapping portion of the shape of the virtual surgical tool 610.

Alternatively, if the overlap processing unit 1410 has determined that there is overlapping between the organ and the virtual surgical tool 610, it can provide the zone coordinate information of the organ to the virtual surgical tool generator unit 720 or request that the virtual surgical tool generator unit 720 read the corresponding information from the storage unit, in order that the virtual surgical tool generator unit 720 may not generate the virtual surgical tool information for the overlapping portion.

The contact recognition unit 1420 may use the virtual surgical tool information generated by the virtual surgical tool generator unit 720 and the zone coordinate information of the organ recognized by the picture analyzer unit 920 to determine whether or not there is contact, and may provide processing correspondingly. If surface coordinate information, from among the organ's zone coordinate information, agrees with the coordinate information of a portion or all of the virtual surgical tool, then it can be determined that there is contact at the corresponding portion. If it is determined by the contact recognition unit 1420 that there is contact, the master robot 1 can provide processing such that, for example, the arm manipulation unit 330 is no longer manipulated, or a force feedback is generated through the arm manipulation unit 330, or warning information (e.g. a warning message or/and a warning sound, etc.) is outputted. Components for processing a force feedback or for outputting warning information can be included as components of the master robot 1.

FIG. 16 shows an example of a method of driving the master robot 1 for outputting a virtual surgical tool according to still another embodiment of the invention.

Referring to FIG. 16, in step 1510, the master robot 1 may receive as input from the operator a manipulation of the arm manipulation unit 330.

Then, in step 1520 and step 1530, the master robot 1 may analyze the operator's manipulation information resulting from the manipulation of the arm manipulation unit 330 to generate virtual surgical tool information. The virtual surgical tool information can include, for example, coordinate information regarding the contours or the area of the virtual surgical tool 610 for outputting the virtual surgical tool 610 through the screen display unit 320.

Also, in step 1540 and step 1550, the master robot 1 may receive a laparoscope picture from the slave robot 2, and may analyze the received picture. Analyzing the received picture can be performed, for example, by the picture analyzer unit 920, where the picture analyzer unit 920 can recognize which organ is included in the laparoscope picture.

In step 1560, the master robot 1 may read the zone coordinate information from the storage unit, regarding the organ recognized through the laparoscope picture.

The master robot 1, in step 1570, may use the coordinate information of the virtual surgical tool 610 and the zone coordinate information of the organ to determine whether or not there is an overlapping portion.

If there is an overlapping portion, the master robot 1 in step 1580 may provide processing such that the virtual surgical tool 610 is outputted through the screen display unit 320 with the overlapping portion concealed.

However, if there is no overlapping portion, the master robot 1 in step 1590 may provide processing such that the virtual surgical tool 610 is outputted through the screen display unit 320 with all portions displayed normally.

FIG. 17 illustrates an embodiment for notifying the operator in the event that the virtual surgical tool 610 contacts the patient's organ. As step 1510 through step 1560 of FIG. 17 have already been described with reference to FIG. 16, they will not be described again.

Referring to FIG. 17, in step 1610, the master robot 1 may determine whether or not a portion of or all of the virtual surgical tool 610 is in contact with an organ. The determining of whether or not there is contact between the organ and the virtual surgical tool 610 can be performed, for example, by using the coordinate information for the respective zones.

If there is contact between the virtual surgical tool 610 and an organ, the process may proceed to step 1620, in which the master robot 1 may perform a force feedback processing to notify the operator. As described above, other processing approaches can be applied, such as preventing further manipulation of the am manipulation unit 330 and outputting warning information (e.g. a warning message or/and a warning sound, etc.), for example.

However, if there is no contact between the virtual surgical tool 610 and an organ, then the process may remain in step 1610.

Through the procedures described above, the operator can predict beforehand whether or not the actual surgical tool 460 will be in contact with an organ, so that the surgery can be conducted with greater safety and accuracy.

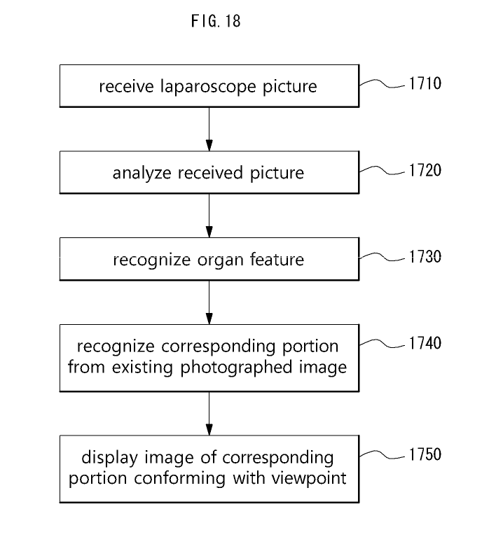

FIG. 18 is a flowchart illustrating a method of providing a reference image according to yet another embodiment of the invention.

Generally, a patient takes various reference pictures, such as X-ray's, CT's, or/and MRI's, etc., before surgery. Presenting such reference pictures together with the laparoscope picture or on a certain monitor of the monitor unit 6 during surgery would enable the operator to perform surgery in a facilitated manner. A corresponding reference picture can be, for example, pre-stored in a storage unit included in the master robot 1 or stored in a database accessible to the master robot 1 over a communication network.

Referring to FIG. 18, in step 1710, the master robot 1 may receive a laparoscope picture from a laparoscope 5 of the slave robot 2.

In step 1720, the master robot 1 may extract preset feature information by using the laparoscope picture. Here, the feature information can include, for example, one or more of an organ's shape within the surgical site, the actual surgical tool's 460 position coordinates, manipulation shape, and the like. Extracting the feature information can also be performed, for example, by the picture analyzer unit 920.

In step 1730, the master robot 1 may use the feature information extracted in step 1720 and other information pre-stored in a storage unit to recognize which organ is being displayed included in the laparoscope picture.

Then, in step 1740, the master robot 1 may read a reference picture, which includes a picture corresponding to the organ recognized in step 1730, from a storage unit or from a database accessible over a communication network, and afterwards may decide which portion of the corresponding reference picture is to be displayed through the monitor unit 6. The reference picture to be outputted through the monitor unit 6 may be a picture taken of the corresponding organ, and can be an X-ray, CT and/or MRI picture, for example. The decision of which portion (e.g. which portion of the corresponding patient's full-body picture) to output for reference can be made based on the name of the recognized organ or the coordinate information of the actual surgical tool 460, and the like. For this purpose, the coordinate information or name of each portion of the reference picture can be specified beforehand, or in the case of reference pictures comprising a series of frames, it can be specified beforehand which frame represents what. The monitor unit 6 can output a single reference picture or display two or more reference pictures together that are different in nature (e.g. an X-ray picture and a CT picture).

In step 1750, the master robot 1 may output the laparoscope picture and the reference picture through the monitor unit 6. Here, providing processing such that the reference picture is displayed in a similar direction to the input angle (e.g. camera angle) of the laparoscope picture can maximize intuitiveness for the operator. For example, if the reference picture is a planar picture taken from a particular direction, then a 3-dimensional picture using real time MPR (multi-planar reformatting) can be outputted according to the camera angle, etc., computed by the characteristic value computation unit 710. MPR is a technique of partially composing a 3-dimensional picture by selectively drawing a certain required portion from one or several slices of sectional pictures, and is more advanced over initial techniques of drawing an ROI (region of interest) one slice at a time.

The foregoing descriptions have been provided focusing on examples in which the master robot 1 operates in a first mode of actual mode, a second mode of compare mode, and/or a third mode of virtual mode. The descriptions that follow will be provided focusing on examples in which the master robot 1 operates in a fourth mode of training mode or a fifth mode of simulation mode. However, the various embodiments related to the display of the virtual surgical tool 610, etc., described above with reference to the related drawings are not intended to be limited to particular drive modes, and can be applied without limitation to any drive mode that requires displaying a virtual surgical tool 610 even when it is not explicitly stated so.

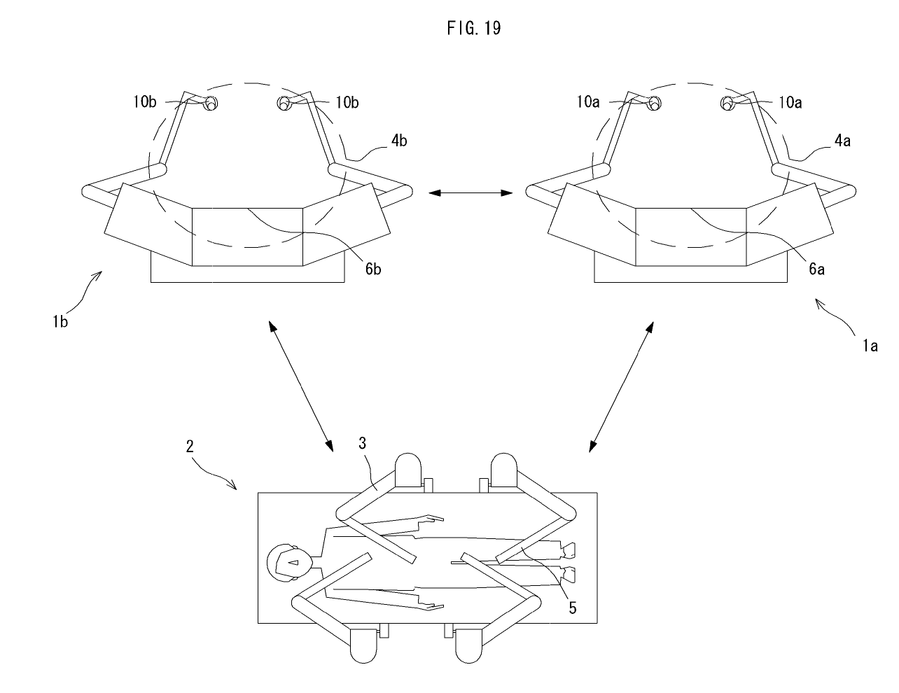

FIG. 19 is a plan view illustrating the overall structure of a surgical robot according to yet another embodiment of the invention.

Referring to FIG. 19, a robot system for laparoscopic surgery may include two or more master robots 1 and a slave robot 2. A first master robot 1a from among the two or more master robots 1 can be a student master robot used by a learner (e.g. a training student), whereas a second master robot 1b can be an instructor master robot used by a trainer (e.g. a training instructor). The compositions of the master robots 1 and the slave robot 2 may be substantially the same as described above and thus will be described briefly.

As described above with reference to FIG. 1, the master interface 4 of a master robot 1 can include a monitor unit 6 and a master controller, while the slave robot 2 can include robot arms 3 and a laparoscope 5. The master interface 4 can further include a mode-changing control button for selecting any one of a multiple number of drive modes. The master controller can be implemented, for example, in a form that can be held by both hands of the operator for manipulation. The monitor unit 6 can output not only the laparoscope picture but also multiple sets of vital information or reference pictures.

In the example shown in FIG. 19, the two master robots 1 can be coupled with each other over a communication network, and each can be coupled with the slave robot 2 over a communication network. The number of master robots 1 coupled with one another over a communication network can vary as needed. Furthermore, while the usage of the first master robot 1a and second master robot 1b, the training instructor and training student can be decided beforehand, the roles can be interchanged with each other as desired or needed.

In one example, the first master robot 1a for a learner can be coupled with only the second master robot 1b for the training instructor over a communication network, while the second master robot 1b can be coupled over a communication network with the first master robot 1a and the slave robot 2. That is, when the training student manipulates the master controller equipped on the first master robot 1a, an arrangement can be provided such that only the virtual surgical tool 610 is manipulated and outputted through the screen display unit 320. Here, the manipulation signal from the first master robot 1a can be provided to the second master robot 1b, and the resulting manipulation of the virtual surgical tool 610 can be outputted through the monitor unit 6b of the second master robot 1b, so that the training instructor may check whether or not the training student performs surgery following normal procedures.

In another example, the first master robot 1a and the second master robot 1b can be coupled with each other over a communication network, with each also coupled with the slave robot 2 over a communication network. In this case, when the training student manipulates the master controller equipped on the first master robot 1a, the actual surgical tool 460 can be manipulated, and a corresponding manipulation signal can be provided also to the second master robot 1b, so that the training instructor may check whether or not the training student performs surgery following normal procedures.

In this case, the training instructor can also manipulate the instructor's own master robot, to control the mode in which the training student's master robot will operate. For this purpose, a certain master robot can be preset such that the drive mode can be decided by a control signal received from another master robot, to enable the manipulation of the actual surgical tool 460 and/or the virtual surgical tool 610.

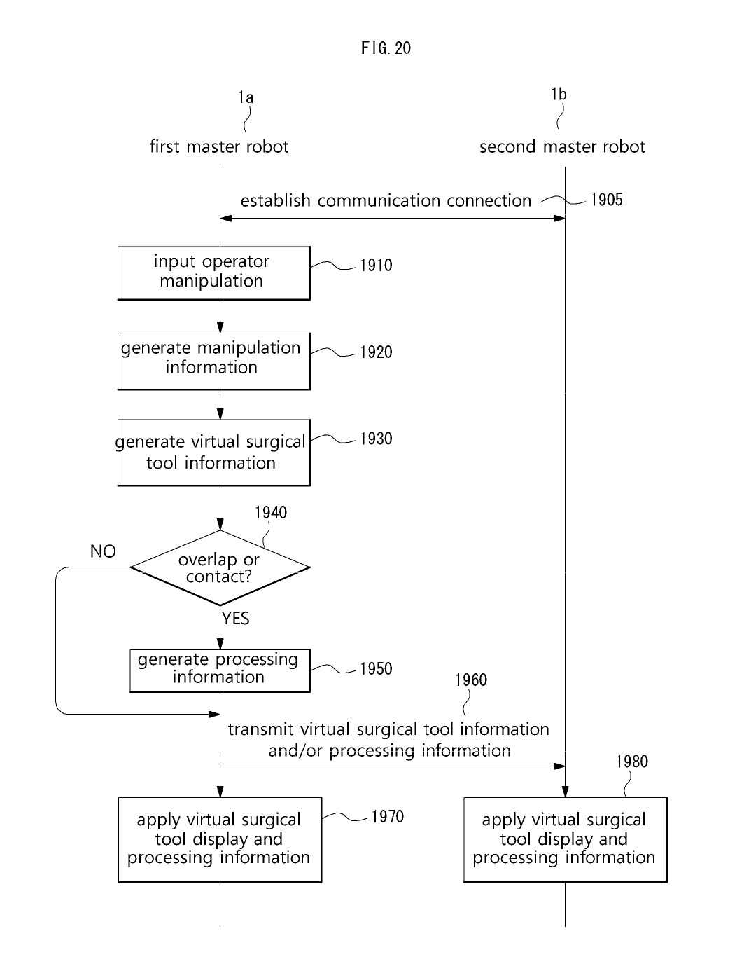

FIG. 20 illustrates a method of operating a surgical robot system in training mode according to yet another embodiment of the invention.

FIG. 20 shows an example of a method of operating a surgical robot system, in which manipulations on the arm manipulation unit 330 of the first master robot 1a serve only to manipulate the virtual surgical tool 610, and the manipulation signals from the first master robot 1a are provided to the second master robot 1b. This can be used when one of the training student and the training instructor applies manipulations on the first master robot 1a and the other of the training student and the training instructor views these manipulations using the second master robot 1b.

Referring to FIG. 20, in step 1905, a communication connection is established between the first master robot 1a and the second master robot 1b. The communication connection can be for exchanging one or more of manipulation signals, authority commands, etc., for example. The communication connection can be established upon a request from one or more of the first master robot 1a and the second master robot 1b, or can also be established immediately when each of the master robots is powered on.

In step 1910, the first master robot 1a may receive a user manipulation according to the manipulation of the arm manipulation unit 330. Here, the user can be, for example, one of a training student and a training instructor.

In step 1920 and step 1930, the first master robot 1a may generate a manipulation signal according to the user manipulation of step 1910, and may generate virtual surgical tool information corresponding to the manipulation signal generated. As described earlier, the virtual surgical tool information can also be generated by using the manipulation information according to the manipulation on the arm manipulation unit 330.

In step 1940, the first master robot 1a may determine whether or not there are overlapping or contacting portions according to the generated virtual surgical tool information. The method of determining whether or not there are overlapping or contacting portions between the virtual surgical tool and an organ has been described above with reference to FIG. 16 and/or FIG. 17, and thus will not be described again.

If there are overlapping or contacting portions, the process may proceed to step 1950, to generate processing information for overlapping or contact. As described above for the examples shown in FIG. 16 and/or FIG. 17, the processing information can include transparency processing for an overlap portion, performing force feedback upon contact, and the like.

In step 1960, the first master robot 1a may transmit virtual surgical tool information and/or processing information to the second master robot 1b. The first master robot 1a can also transmit manipulation signals to the second master robot 1b, and the second master robot 1b can generate virtual surgical tool information using the received manipulation signals, and afterwards determine whether or not there is overlapping or contact.

In step 1970 and step 1980, the first master robot 1a and the second master robot 1b may use the virtual surgical tool information to output a virtual surgical tool 610 on the screen display unit 320. Here, matters pertaining to the processing information can also be processed as well.

The foregoing descriptions have been provided, with reference to FIG. 20, focusing on an example in which the first master robot 1a controls only the virtual surgical tool 610 and the resulting manipulation signals, etc., are provided to the second master robot 1b. However, depending on the drive mode selection, an arrangement can also be provided in which the first master robot 1a controls the actual surgical tool 460 and the resulting manipulation signals, etc., are provided to the second master robot 1b.

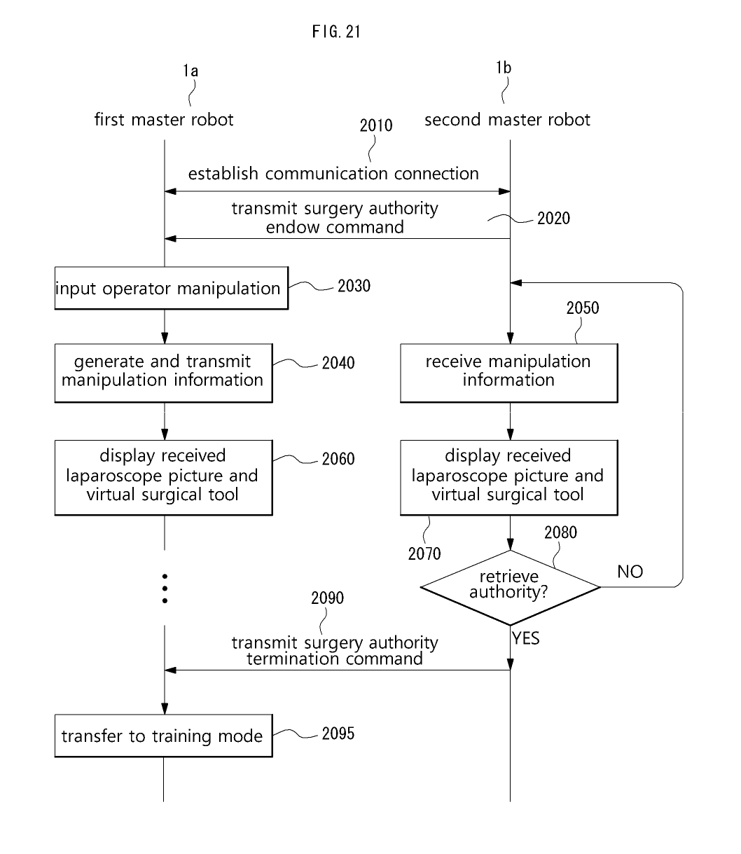

FIG. 21 illustrates a method of operating a surgical robot system in training mode according to yet another embodiment of the invention.

In describing a method of operating a surgical robot system with reference to FIG. 21, an example will be used in which it is assumed that the second master robot 1b has control authority over the first master robot 1a.

Referring to FIG. 21, in step 2010, a communication connection is established between the first master robot 1a and the second master robot 1b. The communication connection can be for exchanging one or more of manipulation signals, authority commands, etc., for example. The communication connection can be established upon a request from one or more of the first master robot 1a and the second master robot 1b, or can also be established immediately when each of the master robots is powered on.

In step 2020, the second master robot 1b may transmit a surgery authority endow command to the first master robot 1a. Upon receiving the surgery authority endow command, the first master robot 1a may obtain the authority to actually control the robot arm 3 equipped on the slave robot 2. The surgery authority endow command can, for example, be generated by the second master robot 1b to be configured in a predefined signal form and information form.

In step 2030, the first master robot 1a may receive as input the user manipulation according to the manipulation of the arm manipulation unit 330. Here, the user can be, for example, a training student.

In step 2040, the first master robot 1a may generate a manipulation signal according to the user manipulation of step 1910 and transmit it over a communication network to the slave robot 2. The first master robot 1a may generate virtual surgical tool information, corresponding to the generated manipulation signal or the manipulation information resulting from the manipulation on the arm manipulation unit 330, so that the virtual surgical tool 610 can be displayed through the monitor unit 6.

Also, the first master robot 1a can transmit the manipulation signal or/and the virtual surgical tool information to the second master robot 1b, to allow checking the manipulation situation of the actual surgical tool 460. In step 2050, the second master robot 1b may receive the manipulation signal or/and virtual surgical tool information.

In step 2060 and step 2070, the first master robot 1a and second master robot 1b may each output the laparoscope picture received from the slave robot 2 and the virtual surgical tool 610 resulting from manipulations on the arm manipulation unit 330 of the first master robot 1a.

In cases where the second master robot 1b is not to output the virtual surgical tool 610 according to the manipulations on the arm manipulation unit 330 of the first master robot 1a through the screen display unit 320 and is to check the manipulation situation of the actual surgical tool 460 through the laparoscope picture received from the slave robot 2, step 2050 can be omitted, and only the received laparoscope picture can be outputted in step 2070.

In step 2080, the second master robot 1b may determine whether or not a request to retrieve the surgery authority endowed to the first master robot 1a is inputted by the user. Here, the user can be, for example, a training student, and can retrieve surgery authority in cases where normal surgery is not being achieved by the user of the first master robot 1a.

If a surgery authority retrieval request is not inputted, the process may again return to step 2050, and the user can observe the manipulation situation of the actual surgical tool 460 by the first master robot 1a.

However, if a surgery authority retrieval request is inputted, in step 2090 the second master robot 1b may transmit a surgery authority termination command over the communication network to the first master robot 1a.

Upon being transmitted the surgery authority termination command, the first master robot 1a can change to the training mode, which allows observing the manipulation situation of the actual surgical tool 460 by the second master robot 1b (step 2095).

The foregoing descriptions have been provided, with reference to FIG. 21, focusing on an example in which the second master robot 1b has control authority over the first master robot 1a. Conversely, however, it is conceivable to have the first master robot 1a transmit the surgery authority termination request to the second master robot 1b.

This may be for transferring authority so that the actual surgical tool 460 can be manipulated by the user of the second master robot 1b, and can be used in situations where the surgery of the corresponding surgical site is difficult or where the surgery of the corresponding surgical site is very easy and is required for training, etc.

Various other measures for transferring surgery authority or control authority between multiple master robots or for endowing/retrieving main authority to/from one master robot can be considered and applied without limitation.

The foregoing descriptions have been provided for various embodiments of the invention with reference to the related drawings. However, the present invention is not limited to the embodiments described above, and various other embodiments can be additionally presented.

According to one embodiment in which multiple master robots are connected over a communication network and are operating in the fourth mode of training mode, an assessment function can also be performed with respect to the learner's ability to control the master robot 1 or perform surgery.

The assessment function of the training mode may be performed during procedures in which the training student manipulates the arm manipulation unit 330 of the second master robot 1b while the training instructor uses the first master robot 1a to conduct surgery. The second master robot 1b may receive a laparoscope picture from the slave robot 2 to analyze characteristic values regarding the actual surgical tool 460 or feature information, and also analyze control process of the virtual surgical tool 610 resulting from the training student's manipulation of the arm manipulation unit 330. Then, the second master robot 1b can evaluate similarities between the movement trajectory and manipulation form of the actual surgical tool 460 included in the laparoscope picture and the movement trajectory and manipulation form of the virtual surgical tool 610 effected by the training student, and thereby calculate an assessment grade for the training student.

According to another embodiment, in the fifth mode of simulation mode, which is an advanced form over the virtual mode, the master robot 1 can also operate as a surgery simulator by coupling the characteristics of an organ with a 3-dimensional shape obtained using a stereo endoscope.

For example, if the liver is included in the laparoscope picture or a virtual screen outputted through the screen display unit 320, the master robot 1 can extract characteristic information of the liver stored in a storage unit and match it with the liver outputted on the screen display unit 320, so that a surgery simulation may be performed in virtual mode during surgery or independent of surgery. The analysis of which organ is included in the laparoscope picture, etc., can be performed by recognizing the color, shape, etc., of the corresponding organ using typical picture processing and recognition technology, and by comparing the recognized information with pre-stored characteristic information. Of course, the decision of which organ is included and/or of which organ the surgery simulation is to be performed for can also be selected by the operator.

In this way, the operator can proceed with a pre-surgery simulation, before actually excising or cutting the liver, to decide how to excise the liver from what direction by using the shape of a liver matched with the characteristic information. During the surgery simulation, the master robot 1 can provide the operator with a tactile feel, regarding whether the portion where a surgical manipulation (e.g. one or more of excision, cutting, suturing, pulling, pushing, etc.) is to be performed is hard or soft, etc., based on the characteristic information (e.g. mathematically modeled information, etc.).

Methods of transferring a corresponding tactile feel may include, for example, performing force feedback processing, adjusting the manipulation sensitivity or manipulation resistance (for example, when pushing the arm manipulation unit 330 forward, a resistive force opposing this push) of the arm manipulation unit 330, and the like.

Also, by having the screen display unit 320 output the section of the organ virtually excised or cut by the operator's manipulation, it is possible to allow the operator to predict the results of an actual excision or cutting.

Also, the master robot 1, in functioning as a surgery simulator, can align an organ's surface shape information, which may be obtained 3-dimensionally using a stereo endoscope, with the organ surface's 3-dimensional shape, which may be reconstructed from a reference picture such as a CT, MRI, etc., and can align an organ interior's 3-dimensional shape, which may be reconstructed from a reference picture, with characteristic information (e.g. mathematically modeled information) through the screen display unit 320, so as to enable the operator to experience a more realistic surgery simulation. The characteristic information can be characteristic information particular to the corresponding patient or can be characteristic information generated for general use.

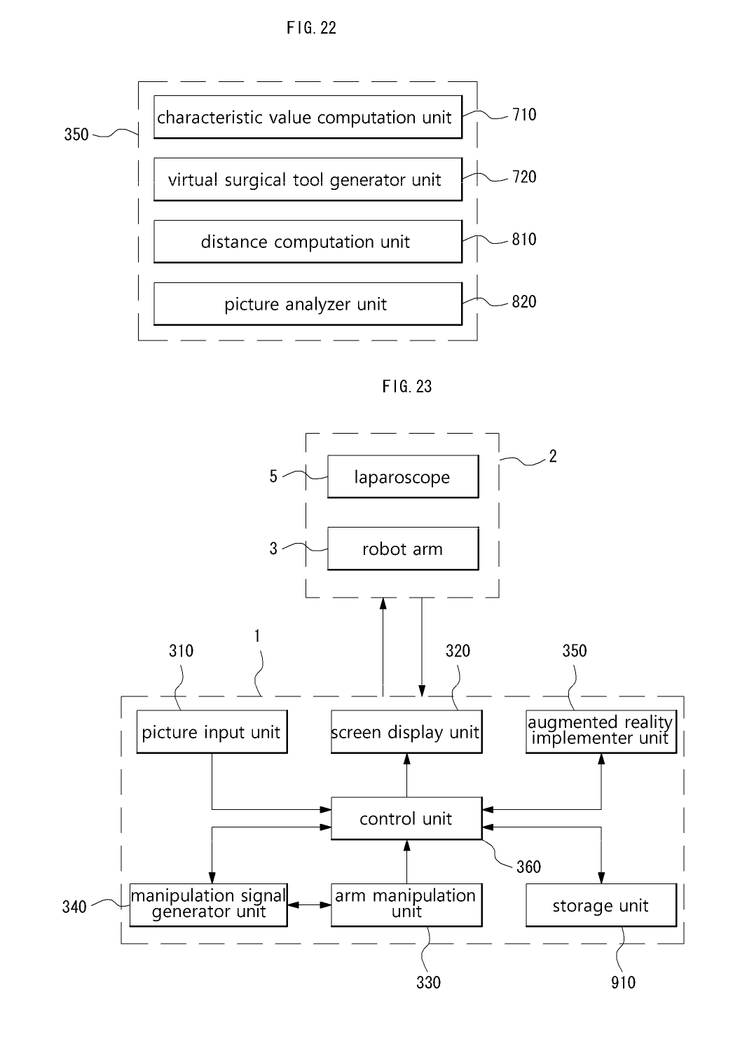

FIG. 22 illustrates the detailed composition of an augmented reality implementer unit 350 according to another embodiment of the invention.

Referring to FIG. 22, the augmented reality implementer unit 350 may include a characteristic value computation unit 710, a virtual surgical tool generator unit 720, a distance computation unit 810, and a picture analyzer unit 820. Some of the components of the augmented reality implementer unit 350 can be omitted, while some components (e.g. a component for processing the vital information received from the slave robot 2 such that it can be outputted through the screen display unit 320, and the like) can be added. One or more of the components included in the augmented reality implementer unit 350 can also be implemented in the form of a software program composed of a combination of program codes.

The characteristic value computation unit 710 may compute the characteristic values by using the picture inputted and provided by the laparoscope 5 of the slave robot 2 and/or coordinate information regarding the position of the actual surgical tool coupled to the robot arm 3. The characteristic values can include, for example, one or more of the laparoscope's 5 field of view (FOV), magnifying ratio, viewpoint (e.g. viewing direction), viewing depth, etc., and the actual surgical tool's 460 type, direction, depth, degree of bending, etc.

The virtual surgical tool generator unit 720 may generate the virtual surgical tool 610 that is to be displayed through the screen display unit 320, by referencing the manipulation information resulting from the operator's manipulation of the robot arm 3.

The distance computation unit 810 may use the position coordinates of the actual surgical tool 460 computed by the characteristic value computation unit 710 and the position coordinates of the virtual surgical tool 610 that moves in conjunction with manipulations on the arm manipulation unit 330, to compute the distance between the surgical tools. For example, when the position coordinates of the virtual surgical tool 610 and the actual surgical tool 460 are decided, the length of the line segment connecting the two points can be computed. Here, the position coordinates can be, for example, the coordinate values of a point in 3-dimensional space defined by the x-y-z axes, and a corresponding point can be pre-designated to be a point at a particular position on the virtual surgical tool 610 and the actual surgical tool 460. In addition, obtaining the distance between the surgical tools can also utilize the length of a path or a trajectory generated by the manipulation method. This is because, if a circle is drawn, for example, and a delay exists during the drawing of the circle, then the length of the line segment between the surgical tools may be very small, although the length of the path or trajectory may be as long as the circumference of the circle generated by the manipulation method.

The position coordinates of the actual surgical tool 460, used for computing distance, can be applied as absolute coordinate values or relative coordinate values with respect to a particular point, or the position of the actual surgical tool 460 as displayed through the screen display unit 320 can be coordinatized. Similarly, for the position coordinates of the virtual surgical tool 610 also, the virtual position moved by manipulations on the arm manipulation unit 330 can be applied as absolute coordinates with respect to the initial position of the virtual surgical tool 610, or relative coordinate values computed with respect to a particular point can be used, or the position of the virtual surgical tool 610 as displayed through the screen display unit 320 can be coordinatized. Here, analyzing the position of each surgical tool displayed through the screen display unit 320 can employ feature information obtained by the picture analyzer unit 820 described below.

If the distance between the virtual surgical tool 610 and the actual surgical tool 460 is small or is 0, then the network communication speed can be considered to be adequate, but if the distance is large, then the network communication speed can be considered to be insufficient.

Using the distance information computed by the distance computation unit 810, the virtual surgical tool generator unit 720 can decide on one or more issues of whether or not to display the virtual surgical tool 610, and the color, form, etc., in which the virtual surgical tool 610 is to be displayed in. For example, if the distance between the virtual surgical tool 610 and the actual surgical tool 460 is equal to or smaller than a preset threshold value, it can be made such that the virtual surgical tool 610 is not outputted through the screen display unit 320. Also, if the distance between the virtual surgical tool 610 and the actual surgical tool 460 exceeds the preset threshold value, a processing can be provided such that the operator has a clear recognition of the network communication speed, for example, by adjusting the translucency, distorting the color, or changing the contour thickness of the virtual surgical tool 610, in proportion to the distance. Here, the threshold value can be designated to be a distance value, such as 5 mm, etc., for example.

The picture analyzer unit 820 may extract preset feature information (e.g. one or more of a color value for each pixel, and the actual surgical tool's position coordinates, manipulation shape, etc.) by using the picture inputted and provided by the laparoscope 5. For example, in order to allow immediate countermeasures in the event of an emergency situation (e.g. excessive bleeding, etc.) during surgery, the picture analyzer unit 820 can analyze the color value for each pixel of the corresponding picture to determine whether or not the pixels having a color value representing blood exceed a base value, or determine whether or not an area or region formed by the pixels having a color value representing blood is equal to or greater than a particular size. Also, the picture analyzer unit 820 can capture the display screen of the screen display unit 320, on which the picture inputted by the laparoscope 5 and the virtual surgical tool 610 are displayed, to generate the position coordinates of the respective surgical tools.

A description will be provided below, with reference to the related drawings, on a method of controlling a surgical system using history information.

The master robot 1 can also function as a surgery simulator in virtual mode or simulation mode, by coupling the characteristics of an organ to a 3-dimensional shape obtained using a stereo endoscope. Using a master robot 1 that is functioning as a surgery simulator, the operator can try conducting surgery on a certain organ or on a surgery patient virtually, and during the virtually conducted surgical procedure, the manipulation history of the operator's arm manipulation unit 10 (e.g. a sequential manipulation for excising the liver) may be stored in the storage unit 910 or/and a manipulation information storage unit 1020. Afterwards, when the operator inputs an automatic surgery command that uses the surgical action history information, a manipulation signal according to the surgical action history information can be transmitted sequentially to the slave robot 2 to control the robot arm 3, etc.

For example, if the liver is included in the laparoscope picture or a virtual screen outputted through the screen display unit 320, the master robot 1 can read the characteristic information (e.g. shape, size, texture, tactile feel during excision, etc.) of a 3-dimensionally modeled liver having a 3-dimensional shape stored in the storage unit 910 and match it with the liver outputted on the screen display unit 320, so that a surgery simulation may be performed in virtual mode or in simulation mode. The analysis of which organ is included in the laparoscope picture, etc., can be performed by recognizing the color, shape, etc., of the corresponding organ using typical picture processing and recognition technology, and by comparing the recognized information with pre-stored characteristic information. Of course, the decision of which organ is included and/or of which organ the surgery simulation is to be performed for can also be selected by the operator.

In this way, the operator can proceed with a pre-surgery simulation, before actually excising or cutting the liver, to decide how to excise the liver from what direction by using the shape of a liver matched with the characteristic information. During the surgery simulation, the master robot 1 can provide the operator with a tactile feel, regarding whether the portion where a surgical manipulation (e.g. one or more of excision, cutting, suturing, pulling, pushing, etc.) is to be performed is hard or soft, etc., based on the characteristic information (e.g. mathematically modeled information, etc.).

Methods of transferring a corresponding tactile feel may include, for example, performing force feedback processing, adjusting the manipulation sensitivity or manipulation resistance (for example, when pushing the arm manipulation unit 330 forward, a resistive force opposing this push) of the arm manipulation unit 330, and the like.

Also, by having the screen display unit 320 output the section of the organ virtually excised or cut by the operator's manipulation, it is possible to allow the operator to predict the results of an actual excision or cutting.

Also, the master robot 1, in functioning as a surgery simulator, can align an organ's surface shape information, which may be obtained 3-dimensionally using a stereo endoscope, with the organ surface's 3-dimensional shape, which may be reconstructed from a reference picture such as a CT, MRI, etc., and can align an organ interior's 3-dimensional shape, which may be reconstructed from a reference picture, with characteristic information (e.g. mathematically modeled information) through the screen display unit 320, so as to enable the operator to experience a more realistic surgery simulation. The characteristic information can be characteristic information particular to the corresponding patient or can be characteristic information generated for general use.

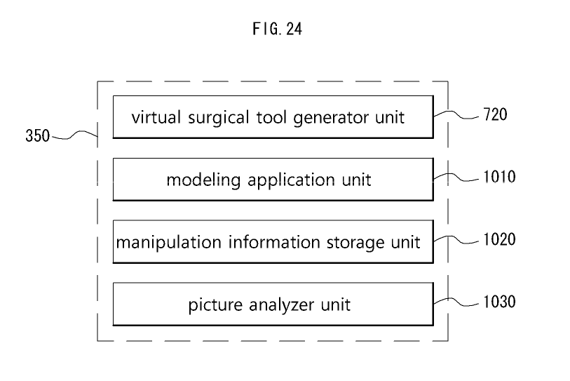

FIG. 23 is a block diagram schematically illustrating the composition of a master robot and a slave robot according to yet another embodiment of the invention, and FIG. 24 illustrates the detailed composition of an augmented reality implementer unit 350 according to yet another embodiment of the invention.

Referring to FIG. 23, which schematically depicts the compositions of the master robot 1 and the slave robot 2, the master robot 1 may include a picture input unit 310, a screen display unit 320, an arm manipulation unit 330, a manipulation signal generator unit 340, an augmented reality implementer unit 350, a control unit 360, and a manipulation information storage unit 910. The slave robot 2 may include a robot arm 3 and a laparoscope 5.

The picture input unit 310 may receive, over a wired or a wireless network, a picture inputted through a camera equipped on the laparoscope 5 of the slave robot 2.

The screen display unit 320 may output an on-screen image, which corresponds to a picture received through the picture input unit 310 and/or the virtual surgical tool 610 according to manipulations on the arm manipulation unit 330, as visual information.

The arm manipulation unit 330 may enable the operator to manipulate the position and function of the robot arm 3 of the slave robot 2.

When the operator manipulates the arm manipulation unit 330 in order to move the position of the robot arm 3 and/or the laparoscope 5 or to perform a manipulation for surgery, the manipulation signal generator unit 340 may generate a corresponding manipulation signal and transmit it to the slave robot 2.

Also, when an instruction is received from the control unit 360 to control the surgical robot system using history information, the manipulation signal generator unit 340 may sequentially generate manipulation signals corresponding to the surgical action history information stored in the storage unit 910 or the manipulation information storage unit 1020 and transmit the manipulation signals to the slave robot 2. The series of procedures for sequentially generating and transmitting the manipulation signals corresponding to surgical action history information can be stopped by the operator inputting a stop command, as described later. Alternatively, instead of sequentially generating and transmitting the manipulation signals, the manipulation signal generator unit 340 can compose one or more sets of manipulation information for multiple surgical actions included in the surgical action history information and transmit these to the slave robot 2.

The augmented reality implementer unit 350 may provide the processing that enables the screen display unit 320 to display not only the picture of the surgical site inputted through the laparoscope 5 and/or a virtual organ modeling image, but also the virtual surgical tool, which moves in conjunction with manipulations on the arm manipulation unit 330 in real time, when the master robot 1 is driven in a virtual mode, simulation mode, etc.

Referring to FIG. 24, which illustrates an example of the augmented reality implementer unit 350, the augmented reality implementer unit 350 can include a virtual surgical tool generator unit 720, a modeling application unit 1010, a manipulation information storage unit 1020, and a picture analyzer unit 1030.

The virtual surgical tool generator unit 720 may generate the virtual surgical tool 610 that is to be displayed through the screen display unit 320, by referencing the manipulation information resulting from the operator's manipulation of the robot arm 3. The position at which the virtual surgical tool 610 is initially displayed can be based, for example, on the display position at which the actual surgical tool 460 is displayed through the screen display unit 320, and the movement displacement of the virtual surgical tool 610 manipulated according to the manipulation on the arm manipulation unit 330 can, for example, be set beforehand by referencing measured values by which the actual surgical tool 460 moves in correspondence with the manipulation signals.

The virtual surgical tool generator unit 720 can also generate only the virtual surgical tool information (e.g. the characteristic values for expressing the virtual surgical tool) for outputting the virtual surgical tool 610 through the screen display unit 320. In deciding the shape or position of the virtual surgical tool 610 according to the manipulation information, the virtual surgical tool generator unit 720 can also reference the characteristic values computed by the characteristic value computation unit 710 or the characteristic values used immediately before for expressing the virtual surgical tool 610.

The modeling application unit 1010 may provide processing such that the characteristic information stored in the storage unit 910 (i.e. characteristic information of a 3-dimensionally modeled image of an organ, etc., inside the body, including for example one or more of interior/exterior shape, size, texture, tactile feel during excision, section and interior shape of an organ excised along an excision direction, and the like) is aligned with the surgery patient's organ. Information on the surgery patient's organ can be recognized by using various reference pictures such as X-rays, CT's, or/and MRI's, etc., taken of the corresponding patient before surgery, and additional information produced by certain medical equipment can be used in correspondence with the reference pictures.

If the characteristic information stored in the storage unit is such that is generated for the body and organs of a person having an average height, the modeling application unit 1010 can scale or transform the corresponding characteristic information according to the reference picture and/or related information. Also, settings related to tactile feel during excision, for example, can be applied after being renewed according to the progression of the corresponding surgery patient's disease (e.g. terminal stage of liver cirrhosis, etc.).

The manipulation information storage unit 1020 may store information on the manipulation history of the arm manipulation unit 10 during a virtual surgical procedure using a 3-dimensionally modeled image. The information on the manipulation history can be stored in the manipulation information storage unit 1020 by the operation of the control unit 360 or/and the virtual surgical tool generator unit 720. The manipulation information storage unit 1020 can be use as a temporary storage space, so that when the operator modifies or cancels a portion of a surgical procedure on the 3-dimensionally modeled image (e.g. modifying the direction of excising the liver, etc.), the corresponding information can be stored together or the corresponding information can be deleted from the stored surgical action manipulation history. In cases where the modify/cancel information is stored together with the surgical action manipulation history, the surgical action manipulation history can be stored with the modify/cancel information applied, when it is moved and stored in the storage unit 910.

The picture analyzer unit 1030 may extract preset feature information (e.g. one or more of a color value for each pixel, and the actual surgical tool's 460 position coordinates, manipulation shape, etc.) by using the picture inputted and provided by the laparoscope 5.

From the feature information extracted by the picture analyzer unit 1030, it is possible, for example, to recognize which organ is currently displayed, as well as to allow immediate countermeasures in the event of an emergency situation (e.g. excessive bleeding, etc.) during surgery. For this purpose, the color value for each pixel of the corresponding picture can be analyzed to determine whether or not the pixels having a color value representing blood exceed a base value, or to determine whether or not an area or region formed by the pixels having a color value representing blood is equal to or greater than a particular size. Also, the picture analyzer unit 1030 can capture the display screen of the screen display unit 320, on which the picture inputted by the laparoscope 5 and the virtual surgical tool 610 are displayed, to generate the position coordinates of the respective surgical tools.

Referring again to FIG. 23, the storage unit 910 may store the characteristic information (e.g. one or more of interior/exterior shape, size, texture, tactile feel during excision, section and interior shape of an organ excised along an excision direction, and the like) of a 3-dimensionally modeled liver having a 3-dimensional shape. Also, the storage unit 910 may store the surgical action history information obtained when the operator conducts virtual surgery using a virtual organ in virtual mode or simulation mode. The surgical action history information can also be stored in the manipulation information storage unit 1020, as already described above. Also, the control unit 360 and/or virtual surgical tool generator unit 720 can further store treatment requirements for an actual surgical procedure or progress information for a virtual surgical procedure (e.g. length, area, shape, bleeding amount, etc., of an incision surface) in the manipulation information storage unit 1020 or the storage unit 910.

The control unit 360 may control the actions of each of the component parts so that the functions described above may be implemented. The control unit 360 can also perform various additional functions, as described in examples for other embodiments.

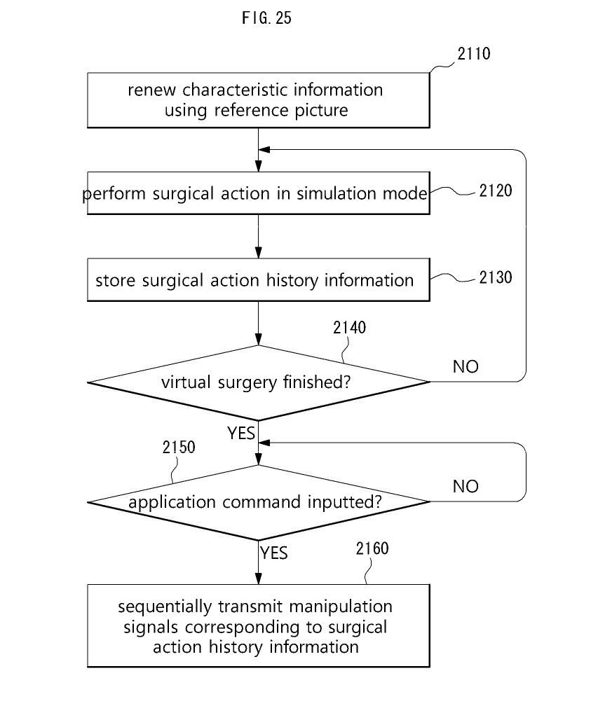

FIG. 25 is a flowchart illustrating a method of automatic surgery using history information according to an embodiment of the invention.

Referring to FIG. 25, in step 2110, the modeling application unit 1010 may, using a reference picture and/or related information, renew the characteristic information of the 3-dimensionally modeled image stored in the storage unit 910. Here, the selection of which virtual organ is to be displayed through the screen display unit 320 can be made, for example, by the operator. Also, the characteristic information stored in the storage unit 910 can be renewed to conform with the actual size, etc., of the surgery patient's organ recognized from the surgery patient's reference picture, etc.

In step 2120 and step 2130, virtual surgery may be conducted by the operator in simulation mode (or virtual mode, the same applies hereinafter), and each procedure of the virtual surgery may be stored as surgical action history information in the manipulation information storage unit 1020 or the storage unit 910. Here, the operator would perform virtual surgery (e.g. cutting, suturing, etc.) on a virtual organ by manipulating the arm manipulation unit 10. Also, treatment requirements for an actual surgical procedure or progress information for a virtual surgical procedure (e.g. length, area, shape, bleeding amount, etc., of an incision surface) can further be stored in the manipulation information storage unit 1020 or the storage unit 910.

In step 2140, it may be determined whether or not the virtual surgery is finished. The finish of the virtual surgery can also be recognized, for example, by the operator inputting a surgery finish command.

If the virtual surgery has not been finished, the process may again proceed to step 2120, otherwise the process may proceed to step 2150.

In step 2150, it may be determined whether or not an application command is inputted for controlling a surgery system using surgical action history information. Before proceeding with automatic surgery according to the input of the application command in step 2150, a confirmation simulation and a complementary process can be performed by the operator to check whether the stored surgical action history information is suitable. That is, it can be arranged such that, after a command is provided to proceed with automatic surgery according to surgical action history information in virtual mode or simulation mode, and the operator checks the automatic surgery procedure on a screen, if there are aspects that are insufficient or require improvement, the application command of step 2150 is inputted after complementing such aspects (i.e. renewing the surgical action history information).

If the application command has not been inputted, the process may remain at step 2150, otherwise the process may proceed to step 2160.

In step 2160, the manipulation signal generator unit 340 may sequentially generate manipulation signals corresponding to the surgical action history information stored in the storage unit 910 or the manipulation information storage unit 1020 and may transmit the manipulation signals to the slave robot 2. The slave robot 2 would sequentially proceed with surgery on the surgery patient in correspondence to the manipulation signals.

The example in FIG. 25 described above is for such cases where the operator performs virtual surgery to store surgical action history information and afterwards uses this to control the slave robot 2.

The procedures of step 2110 through step 2140 can be for an entire surgical procedure, where the surgery on a surgery patient is initiated and finished completely, or can be for a partial procedure of a partial surgical step.

A partial procedure can relate to a suturing motion, for example, such that when a pre-designated button is pressed while holding a needle in the vicinity of the suturing site, the needle can be sewn and a knot can be tied automatically. Alternatively, according to the set preferences, the partial procedure can be performed only up to the point of sewing the needle and tying the knot, with the operator performing the remaining procedures directly.

Another example involving a dissection motion can include a first robot arm and a second robot arm holding an incision site, and when the operator steps on a pedal, a treatment can be processed automatically as a partial procedure such that the portion in-between can be cut by a pair of scissors or by a monopolar, etc.

In such cases, while the automatic surgery is conducted according to surgical action history information, the automatic surgery can be kept at a halted state (e.g. holding) until the operator performs a designated action (e.g. stepping on the pedal), and the next step of the automatic surgery can be continued when the designated action is completed.

As such, an incision can be made in the skin, etc., while the holding of the tissue is switched between both hands and with manipulations made by the foot, so that the surgery can be conducted with greater safety, and various treatments can be applied simultaneously with a minimum number of operating staff.

It is also possible to further subdivide and categorize each surgical action (e.g. basic actions such as suturing, dissecting, etc.) into unit actions and create an interrelated action map, so that selectable unit actions can be listed on a user interface (UI) of the display unit. In this case, the operator can select an appropriate unit action by using an easy method of selection such as scrolling, clicking, etc., to perform the automatic surgery. When one unit action is selected, the operator can easily select the next action, and by repeating this process, the automatic surgery can be performed for a desired surgical action. Here, the surgeon can choose the direction and position of instruments to be suitable for the corresponding action, and can initiate and perform the automatic surgery. The surgical action history information for the partial actions and/or unit actions described above can be stored beforehand in a certain storage unit.

Also, while the procedures of step 2110 through step 2140 can be performed during surgery, it is possible to complete the steps before surgery and have the corresponding surgical action history information stored in the storage unit 910, and the operator can perform the corresponding action simply by selecting which partial action or entire action to perform and inputting an application command.

As described above, this embodiment can subdivide the operating steps of automatic surgery to prevent undesired results, and can adapt to the various environments which the body tissues may be in for the subject of surgery. Also, in cases of simple surgical actions or typical surgical actions, several actions can be grouped together, according to the judgment of the surgeon, to be selected and performed, so that the number of selection steps may be reduced. For this purpose, an interface for selection, such as a scroll or a button, etc., can be provided on the grip portions of the operator's console, and a display user interface that enables easier selection can also be provided.

As such, the surgery function using surgical action history information according to the present embodiment can be used not only as part of a method of performing automatic surgery using augmented reality, but also as a method of performing automatic surgery without using augmented reality if necessary.

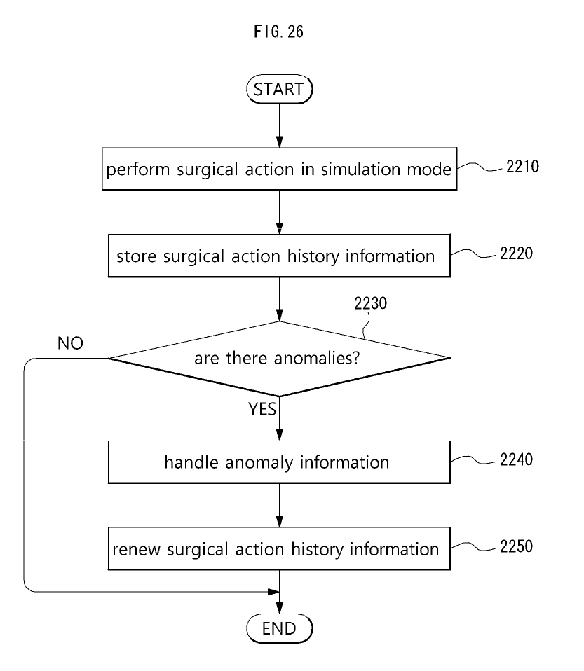

FIG. 26 is a flowchart illustrating a procedure of renewing surgical action history information according to another embodiment of the invention.

Referring to FIG. 26, in step 2210 and step 2220, virtual surgery may be conducted by the operator in simulation mode (or virtual mode, the same applies hereinafter), and each procedure of the virtual surgery may be stored as surgical action history information in the manipulation information storage unit 1020 or the storage unit 910. Also, treatment requirements for an actual surgical procedure or progress information for a virtual surgical procedure (e.g. length, area, shape, bleeding amount, etc., of an incision surface) can further be stored in the manipulation information storage unit 1020 or the storage unit 910.

In step 2230, the control unit 360 may determine whether or not there are anomalies in the surgical action history information. For example, there can be cancellations or modifications in certain procedures of the operator's surgical procedures using a 3-dimensionally modeled image, and shaking of the virtual surgical tool caused by shaky hands on the operator, unnecessary movement paths in moving the position of the robot arm 3, and the like.

If there is an anomaly, the corresponding anomaly can be handled in step 2240, after which the process may proceed to step 2250 to renew the surgical action history information. For example, if there was a cancellation or modification of some of the procedures in the surgical procedure, processing can be provided to remove this from the surgical action history information, so that the corresponding process is not actually performed by the slave robot 2. Also, if there was shaking of the virtual surgical tool caused by shaky hands on the operator, a correction can be applied such that the virtual surgical tool is moved and manipulated without shaking, so that the control of the robot arm 3 can be more refined. Also, if there were unnecessary movement paths in moving the position of the robot arm 3, that is, if after a surgical manipulation at position A, there was movement to positions B and C for no reason, and then another surgical manipulation at position D, then the surgical action history information can be renewed to have direct movement from position A to position D, or the surgical action history information can be renewed to have the movement from A through D approximates a curve.

The surgical action history information for step 2220 and step 2250 described above can be stored in the same storage space. However, it is also possible to have the surgical action history information for step 2220 stored in the manipulation information storage unit 1020 and have the surgical action history information for step 2250 stored in the storage unit 910.

Also, the procedures for processing anomalies in step 2230 through step 2250 described above can be processed at the time when the surgical action history information is stored in the manipulation information storage unit 1020 or storage unit 910, or can be processed before the manipulation signal generator unit 340 generates and transmits the manipulation signal.

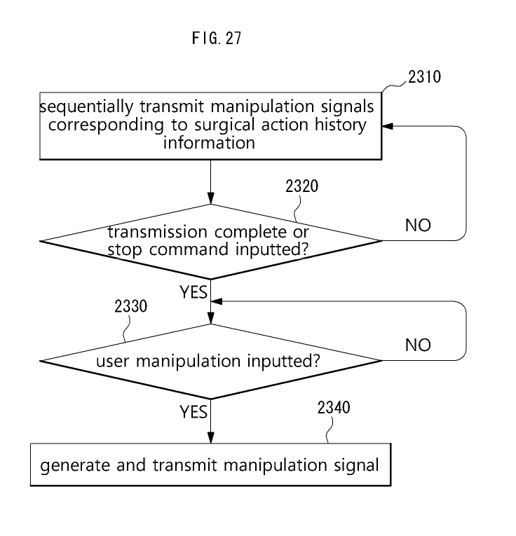

FIG. 27 is a flowchart illustrating a method of automatic surgery using history information according to yet another embodiment of the invention.

Referring to FIG. 27, in step 2310, the manipulation signal generator unit 340 may sequentially generate manipulation signals corresponding to the surgical action history information stored in the storage unit 910 or the manipulation information storage unit 1020 and may transmit the manipulation signals to the slave robot 2. The slave robot 2 would sequentially conduct surgery on the surgery patient in correspondence to the manipulation signals.

In step 2320, the control unit 360 may determine whether or not the generation and transmission of manipulation signals by the manipulation signal generator unit 340 have been completed or a stop command has been inputted by the operator. For example, the operator can input a stop command if there is a discrepancy between a situation in virtual surgery and a situation in the actual surgery performed by the slave robot 2, or if an emergency situation has occurred, and so on.

If transmission has not been completed or a stop command has not been inputted, the process may again proceed to step 2310, otherwise the process may proceed to step 2330.

In step 2330, the master robot 1 may determine whether or not a user manipulation has been inputted that uses one or more of the arm manipulation unit 330, etc.

If the user manipulation is inputted, the process may proceed to step 2340, otherwise the process may remain at step 2330.

In step 2340, the master robot 1 may generate a manipulation signal according to the user manipulation and transmit the manipulation signal to the slave robot 2.

In the example shown in FIG. 27 described above, during automatic operation of an entire or partial surgical procedure using history information, the operator can input a stop command to perform a manipulation manually, and afterwards return again to automatic surgery. In this case, the operator can output on the screen display unit 320 the surgical action history information stored in the storage unit 910 or manipulation information storage unit 1020, delete portions of manual manipulation or/and portions requiring deletion, and then proceed again for subsequent procedures beginning at step 2310.

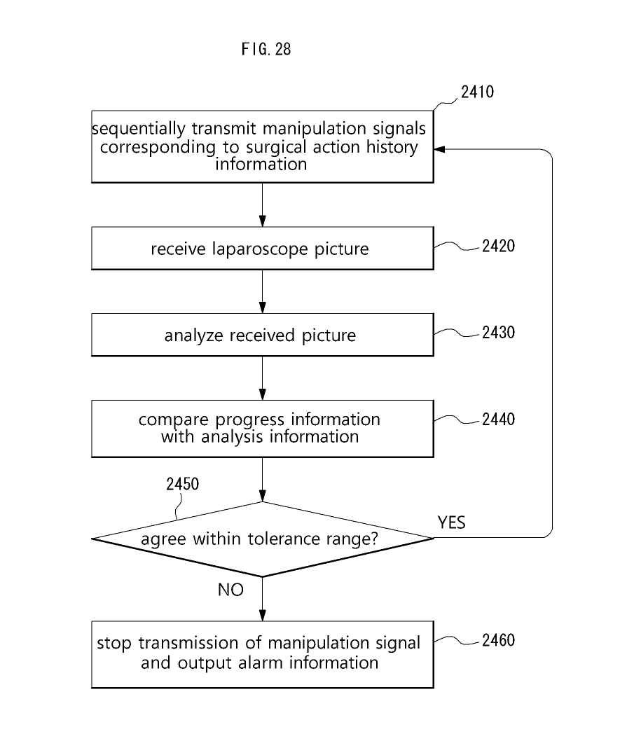

FIG. 28 is a flowchart illustrating a method of monitoring surgery progress according to yet another embodiment of the invention.

Referring to FIG. 28, in step 2410, the manipulation signal generator unit 340 may sequentially generate manipulation signals according to the surgical action history information and transmit the manipulation signals to the slave robot 2.

In step 2420, the master robot 1 may receive a laparoscope picture from the slave robot 2. The received laparoscope picture would be outputted through the screen display unit 320, and the laparoscope picture can include depictions of the surgical site and the actual surgical tool 460 being controlled according to the sequentially transmitted manipulation signals.

In step 2430, the picture analyzer unit 1030 of the master robot 1 may generate analysis information, which is an analysis of the received laparoscope picture. The analysis information can include, for example, a length, area, shape, and bleeding amount of an incision surface. The length or area of an incision surface can be analyzed, for example, by way of picture recognition technology such as of extracting the contours of an object within the laparoscope picture, while the bleeding amount can be analyzed by computing the color value for each pixel in the corresponding picture and evaluating the area or region, etc., of the pixels targeted for evaluation. The picture analysis based on picture recognition technology can be performed, for example, by the characteristic value computation unit 710.

In step 2440, the control unit 360 or the picture analyzer unit 1030 may compare progress information (e.g. a length, area, shape, and bleeding amount of an incision surface), which may be generated during a virtual surgical procedure and stored in the storage unit 910, with the analysis information generated through step 2430.

In step 2450, it may be determined whether or not the progress information and the analysis information agree with each other within a tolerance range. The tolerance range can be pre-designated, for example, as a particular ratio or difference value for each comparison item.

If the two agree within the tolerance range, the process may proceed to step 2410 to repeat and perform the procedures described above. Of course, the automatic surgery procedure can obviously be stopped as described above by the operator's stop command, etc.

However, if the two do not agree within the tolerance range, the process may proceed to step 2460, in which the control unit 360 may provide control such that the generation and transmission of manipulation signals according to the surgical action history information is stopped, and alarm information may be outputted through the screen display unit 320 and/or a speaker unit. Because of the outputted alarm information, the operator can recognize occurrences of emergency situations or situations having discrepancies from virtual surgery and thus respond immediately.

The method of controlling a laparoscopic surgical robot system using augmented reality and/or history information as described above can also be implemented as a software program, etc. The code and code segments forming such a program can readily be inferred by computer programmers of the relevant field of art. Also, the program can be stored in a computer-readable medium and can be read and executed by a computer to implement the above method. The computer-readable medium may include magnetic storage media, optical storage media, and carrier wave media.

While the present invention has been described with reference to particular embodiments, it is to be appreciated that various changes and modifications can be made by those skilled in the art without departing from the spirit and scope of the present invention as defined by the scope of claims set forth below.

1-32. (canceled)

33. A method of controlling a surgical robot system, the method performed in a master robot, the master robot configured to control a slave robot having a robot arm, the method comprising:
displaying an endoscope picture corresponding to a picture signal inputted from a surgical endoscope;
receiving as input manipulation information according to a manipulation on the arm manipulation unit;
generating the virtual surgical tool information and a manipulation signal for controlling the robot arm according to the manipulation information; and
displaying a virtual surgical tool corresponding to the virtual surgical tool information together with the endoscope picture,
wherein the manipulation signal is transmitted to the slave robot for controlling the robot arm.

34-35. (canceled)

36. The method according to claim 33, further comprising:
receiving as input a drive mode selection command for designating a drive mode of the master robot; and
providing control such that one or more of the endoscope picture and the virtual surgical tool are displayed through the screen display unit according to the drive mode selection command.

37-38. (canceled)

39. The method according to claim 33, further comprising:
receiving vital information measured from the slave robot; and
displaying the vital information in a display area independent of a display area having the endoscope picture displayed thereon.

40. The method according to claim 33, further comprising:
computing a characteristic value using one or more of the endoscope picture and position coordinate information of an actual surgical tool coupled to the robot arm,
wherein the characteristic value includes one or more of the surgical endoscope's field of view, magnifying ratio, viewpoint, and viewing depth, and the actual surgical tool's type, direction, depth, and bent angle.

41. The method according to claim 33, further comprising:
transmitting a test signal to the slave robot;
receiving a response signal in response to the test signal from the slave robot; and
calculating a delay value for one or more of a network communication speed and a network communication delay time between the master robot and the slave robot by using a transmission time of the test signal and a reception time of the response signal.

42. The method according to claim 41, wherein the displaying of the virtual surgical tool together with the endoscope picture comprises:
determining whether or not the delay value is equal to or lower than a preset delay threshold value;
providing processing such that the virtual surgical tool is displayed together with the endoscope picture, if the delay threshold value is exceeded; and
providing processing such that only the endoscope picture is displayed, if the delay threshold value is not exceeded.

43. The method according to claim 33, further comprising:
computing position coordinates of the endoscope picture displayed including an actual surgical tool and the displayed virtual surgical tool; and
computing a distance value between the respective surgical tools by using the position coordinates of the respective surgical tools.

44. The method according to claim 43, wherein the displaying of the virtual surgical tool together with the endoscope picture comprises:
determining whether or not the distance value is equal to or lower than a preset distance threshold value; and
providing processing such that the virtual surgical tool is displayed together with the endoscope picture only if the distance value is equal to or lower than the distance threshold value.

45. The method according to claim 43, wherein the displaying of the virtual surgical tool together with the endoscope picture comprises:
determining whether or not the distance value is equal to or lower than a preset distance threshold value; and
providing processing such that the virtual surgical tool is displayed together with the endoscope picture, with one or more processing for adjusting translucency, changing color, and changing contour thickness applied to the virtual surgical tool, if the distance threshold value is exceeded.

46. The method according to claim 43, further comprising:
determining whether or not the position coordinates of each of the surgical tools agree with each other within a tolerance range; and
verifying a communication status between the master robot and the slave robot from a result of the determining.

47. The method according to claim 46, wherein the determining comprises:
determining whether or not current position coordinates of the virtual surgical tool agree with previous position coordinates of the actual surgical tool within a tolerance range.

48. The method according to claim 46, wherein the determining further comprises:
determining whether or not one or more of a trajectory and manipulation type of each of the surgical tools agree with each other within a tolerance range.

49. The method according to claim 33, further comprising:
extracting feature information, the feature information containing a color value for each pixel in the endoscope picture being displayed;
determining whether or not an area or a number of pixels in the endoscope picture having a color value included in a preset color value range exceeds a threshold value, and
outputting warning information if the threshold value is exceeded.

50. (canceled)

51. The method according to claim 33, wherein the displaying of the virtual surgical tool together with the endoscope picture comprises:
extracting zone coordinate information of a surgical site or an organ displayed through the endoscope picture by way of image processing the endoscope picture;
determining by using the virtual surgical tool information and the zone coordinate information whether or not there is overlapping such that the virtual surgical tool is positioned behind the zone coordinate information; and
providing processing such that a portion of a shape of the virtual surgical tool where overlapping occurs is concealed, if there is overlapping.

52. The method according to claim 33, further comprising:
extracting zone coordinate information of a surgical site or an organ displayed through the endoscope picture by way of image processing the endoscope picture;
determining by using the virtual surgical tool information and the zone coordinate information whether or not there is contact between the virtual surgical tool and the zone coordinate information; and
performing processing such that a contact warning is provided, if there is contact.

53. (canceled)

54. The method according to claim 33, comprising:
recognizing a surgical site or an organ displayed through the endoscope picture, by way of image processing the endoscope picture; and
extracting and displaying a reference picture of a position corresponding to a name of the recognized organ from among pre-stored reference pictures,
wherein the reference picture includes one or more of an X-ray picture, a computed tomography (CT) picture, and a magnetic resonance imaging (MRI) picture.

55. The method according to claim 40, comprising:
extracting a reference image corresponding to the position coordinates of the actual surgical tool from among pre-stored reference pictures; and
extracting and displaying the extracted reference picture,
wherein the reference picture includes one or more of an X-ray picture, a computed tomography (CT) picture, and a magnetic resonance imaging (MRI) picture.

56. (canceled)

57. The method according to claim 54, wherein the reference picture is displayed as a 3-dimensional picture using MPR (multi-planar reformatting).

58. The method according to claim 55, wherein the reference picture is displayed as a 3-dimensional picture using MPR (multi-planar reformatting).

59. A method of operating a surgical robot system, the surgical robot system comprising a slave robot having a robot arm and a master robot controlling the slave robot, the method comprising:
generating, by a first master robot, of virtual surgical tool information for displaying a virtual surgical tool in correspondence with a manipulation on an arm manipulation unit and of a manipulation signal for controlling the robot arm; and
transmitting, by the first master robot, of the manipulation signal to the slave robot and of one or more of the manipulation signal and the virtual surgical tool information to a second master robot,
wherein the second master robot displays a virtual surgical tool corresponding to one or more of the manipulation signal and the virtual surgical tool information through a screen display unit.

60. The method according to claim 59, wherein each of the first master robot and the second master robot displays an endoscope picture received from the slave robot through a screen display unit, and the virtual surgical tool is displayed together with the endoscope picture.

61. The method according to claim 59, further comprising:
determining, by the first master robot, of whether or not a surgery authority retrieve command is received from the second master robot; and
providing control, by the first master robot, such that a manipulation on the arm manipulation unit functions only to generate the virtual surgical tool information, if the surgery authority retrieve command is received.

62-113. (canceled)
